(19) United States
(12) Patent Application Publication
(10) Pub. No.: US 2022/0369957 A1
(43) Pub. Date: Nov. 24, 2022

(54) SELF-CONTAINED DERMAL PATCH FOR BLOOD ANALYSIS

(71) Applicant: SATIO, INC., Weston, MA (US)

(72) Inventor: Namal NAWANA, Weston, MA (US)

(73) Assignee: SATIO, INC., Weston, MA (US)

(21) Appl. No.: 17/747,544

(22) Filed: May 18, 2022

Related U.S. Application Data
(60) Provisional application No. 63/190,700, filed on May 19, 2021.

(51) Int. Cl. A61B 5/15; G01N 33/49

(52) U.S. Cl. CPC A61B 5/1411; A61B 5/150022; A61B 5/150412; A61B 5/150969; G01N 33/491

(57) ABSTRACT

In one aspect, a dermal patch is disclosed, which comprises at least one needle configured for puncturing a subject's skin so as to allow drawing blood from the subject, a first blood-transfer fluidic channel for receiving at least a portion of the drawn blood, and a serum-separation element fluidly coupled to said first blood-transfer fluidic channel for receiving at least a portion of the drawn blood and separating a serum component thereof. In some embodiments, the dermal patch may include at least one reservoir for storing blood-processing reagent(s).

### CROSS REFERENCE TO RELATED APPLICATIONS

This application claims priority to and the benefit of U.S. Application No. 63/190,700 filed on May 19, 2021, the entire teachings of this application are incorporated by reference herein.

### BACKGROUND

The present teachings are generally directed to dermal patches that can be employed to collect a physiological sample from a subject and analyze the sample so as to detect and optionally quantify a variety of target analytes, such as a variety of biomarkers.

Biomarkers are increasingly employed for diagnosis of various disease conditions as well as for assessing treatment protocols. In many cases, it is important to monitor the level of a biomarker over time to assess, e.g., the progression of a disease condition. Such temporal monitoring of biomarkers using conventional techniques for sample collection can be cumbersome and painful for the patient. For example, the invasive nature of drawing a blood sample from a patient can cause discomfort and may lead to less cooperation from a subject, especially children, and hence render multiple measurements of a target analyte difficult.

Some recently-developed applicator devices that allow continuous monitoring of certain analytes, such as glucose, fail to provide a solution for improving the detection and measurement of the levels of a target analyte at a plurality of discrete times in small volumes of a physiological sample (e.g., blood) extracted from a subject. Further, such conventional applicators typically suffer from a number of shortcomings, such as, low sensitivity and/or specificity.

### SUMMARY

In one aspect, a dermal patch is disclosed, which comprises a plurality of sensing units, and a plurality of needles, where each of the needles is associated with one of the sensing units and is configured for puncturing the skin for drawing blood for introduction into the respective sensing unit. At least one serum-separation element is associated with at least one of the sensing units for receiving blood and separating a serum/plasma component of the blood for introduction into said at least one of the sensing units.

In some embodiments, the dermal patch includes a plurality of serum-separation elements, each of which is associated with one of the sensing units.

In some embodiments, the dermal patch can include one or more pumps for facilitating transfer of the blood to one or more sensing units of the dermal patch. For example, in some embodiments, the dermal patch can include a plurality of pumps each of which is configured to facilitate transfer of blood drawn via one of those needles to a sensing unit associated with that needle. A variety of passive and/or active pumps can be employed. Some examples of such pumps can include, without limitation, a capillary pump, a positive displacement pump, among others.

In some embodiments, the serum-separating element includes a fibrous element that is configured to capture one or more cellular components of the blood so as to separate a plasma/serum component of the blood for analysis. For example, in such embodiments, the serum component can be introduced in a respective sensing unit for analysis, e.g., for detection and optionally quantification of one or more biomarkers and/or other analytes.

In some embodiments, the serum-separating element is a fibrous element. For example, in some embodiments, the serum-separating element can be a nitrocellulose strip. The applicant has discovered that the use of such a fibrous element, and in particular a nitrocellulose strip, can allow sufficient fractionation of the blood to enhance significantly the sensitivity/specificity of detection of analytes (e.g., biomarkers) in the separated serum, especially using a graphene-based sensor. In other words, although the use of a nitrocellulose strip in a patch according to some embodiments may not result in fractionation of the whole blood sample with the same degree of separation quality that is achievable via traditional fractionation methods, such as differential centrifugation; nonetheless, the applicant has discovered that the use of such a nitrocellulose strip in embodiments of the dermal patch can significantly enhance the sensitivity/specificity for the detection of a variety of analytes (e.g., biomarkers) using a variety of detectors, such as graphene-based detectors, relative to the use of a whole blood sample for such detection.

A variety of detectors can be incorporated in a dermal patch according to various embodiments. Some examples of such detectors include, without limitation, a graphene-based detector, a chemical detector, a lateral flow sensor, among others.

The dermal patch may include a needle-activation mechanism for selective activation of the needles such that, when in an activated state, each needle can puncture a subject's skin for drawing blood for introduction into a sensing unit that is associated with the activated needle. By way of example, the needle-activation mechanism can include a cam for selectively activating the needles, for example, via activating a spring coupled to that needle (e.g., by causing the compression of the spring to move the needle into a deployed (activated) state).

In some embodiments, each of the plurality of the needles is configured to move between a retracted position and a deployed position, where in the deployed position the needle is capable of puncturing the skin.

In some embodiments, the dermal patch may include at least one reservoir associated with at least one of a plurality of sensing units, where the reservoir is configured to store one or more blood processing reagents, buffers, etc., such as those disclosed herein. In some such embodiments, the reservoir can include a frangible membrane that can be burst to release the reagents contained in the reservoir for mixing with a sample of drawn blood. In some cases, a wicking element can be used to draw the mixture of the blood and the processing reagents into a serum-separation element (e.g., a nitrocellulose strip), which is configured to separate the serum component for introduction into a sensing unit disposed on the patch.

The dermal patch may include a plurality of fluidic channels, each associated with one of the needles, where each of the channels comprises an inlet for receiving a blood sample via a skin puncture generated by the respective needle. Further, each fluidic channel can include an outlet through which at least a portion of the received blood sample can exit the channel. In some such embodiments, the outlet of each channel is positioned so as to deliver the blood exiting the channel to a serum-separating element that is associated with the respective needle. In some embodiments, at least one of the needles can include a channel (e.g., a central channel that extends from the tip of the needle to its base) through which blood can be collected for introduction to one or more sensing units of the dermal patch.

In a related aspect, a dermal patch for collection and analysis of blood from a subject is disclosed, which includes at least one needle configured for puncturing a subject's skin so as to allow drawing blood from the subject, a first blood-transfer fluidic channel for receiving at least a portion of the drawn blood, and a serum-separation element fluidly coupled to the first blood-transfer fluidic channel for receiving at least a portion of the drawn blood and separating a plasma or a serum component thereof for analysis. Although such an element is herein referred to as a serum-separation element, it can be configured to separate the serum component or the plasma component of the blood.

In some embodiments, the serum-separation element includes at least one fibrous membrane configured to capture at least a portion of one or more cellular components of the received blood, thereby separating a serum (or a plasma) component of the blood.

By way of example, the serum-separation element can be formed of nitrocellulose, e.g., it can be a nitrocellulose strip. In some embodiments, prior, during or after the introduction a blood sample into the serum-separation element, one or more anti-coagulant reagents (e.g., heparin) can be added to the sample. In some embodiments, such an anti-coagulant reagent can be incorporated in the serum-separation element.

In some embodiments, the first blood-transfer fluidic channel includes an inlet for receiving at least a portion of the blood drawn from the subject and an outlet through which at least a portion of the received blood exits the fluidic channel.

In some embodiments, the dermal patch can further include at least one blood-analysis chamber having an inlet in fluid communication with the outlet of the fluidic channel for receiving at least a portion of the blood exiting the fluidic channel. A fluidic channel is a channel that allows passage of a fluid (e.g., liquid or gas) therethrough. At least one sensor operably coupled to the blood-analysis chamber can detect at least one analyte in said separated serum component.

A variety of sensors can be employed for the detection of the target analyte. Some examples of such sensors include, without limitation, a graphene-based sensor, a lateral flow immunoassay sensor, a chemical sensor, among others.

In some embodiments, the separated plasma or the serum component can still include some cellular elements. It has been discovered that even without having a level of fractionation that is achieved via traditional methods, such as differential centrifugation, the separated serum component can be utilized to achieve an enhanced detection sensitivity/specificity relative to using whole blood for detecting, and optionally quantifying, a variety of target analytes in a drawn blood sample. Some examples of such target analytes may include, without limitation, a biomarker, such as troponin, brain natriuretic peptide (BnP), or other biomarkers including those disclosed herein.

In some embodiments, the serum-separation element can include at least one wicking element disposed in proximity of the outlet of the serum-separation fluidic channel for capturing at least a portion of the separated serum component and delivering at least a portion of the captured serum component to a sensing element of the sensor via a wicking action.

As noted above, in some embodiments, the separated serum component may include any of a plurality of red blood cells and/or a plurality of white blood cells and/or platelets. However, the concentration of such cellular components in the separated serum component can be less than that in the whole blood by a factor in a range of about 2 to about 2000 (for example about 10 to about 1000, about 50 to about 750, about 100 to about 500, etc.), though lower concentrations can also be achieved.

In some embodiments, the dermal patch can include a housing for containing various components of the dermal patch. The housing can be formed of a variety of suitable materials, such as a variety of polymeric materials, e.g., PDMS (poly(dimethylsiloxane)). The housing can be formed as a single unit or multiple units that are coupled to one another. In some embodiments, the housing can include one or more windows to allow visualization of one or more internal components of the dermal patch, e.g., blood sample chambers. In some embodiments, an adhesive layer can be used for attaching the dermal patch to a subject's skin.

In some embodiments, the housing can include a cap and a base, which can be releasably coupled to one another. In some cases, the base can be attached to the subject's skin, e.g., via an adhesive membrane, and the cap can be coupled to the base, e.g., to activate the collection of one or more blood samples and introduce the blood drawn from the subject into sensing units of the dermal patch. In some embodiments, the cap can be removed and the base can be maintained on the subject's skin.

In some embodiments, the dermal patch can include a plurality of microneedles. In some embodiments, the microneedle(s) can be moved between a retracted position, where the microneedle(s) are moved into an enclosure provided in the housing to a deployed position in which the microneedles are at least partially exposed for puncturing a subject's skin.

In some such embodiments, the whole-blood receiving well can include at least one transparent window, e.g., to view a lateral flow sensor, to determine whether the sensor has detected the presence of an analyte of interest in the drawn blood. In some embodiments, the blood received in the well is in the form of a monolayer.

In some embodiments, the dermal patch includes a blood distribution fluidic device having an inlet in fluid communication with the first blood transfer fluidic channel for receiving at least a portion of the drawn blood, said blood distribution fluidic device having at least two outlets one of which is in fluid communication with said at least one well and another one of which is in fluidic communication with the serum-separation element for distributing the received blood between said at least one well and said serum-separation element.

In a related aspect, a dermal patch is disclosed, which comprises a plurality of microneedles configured for puncturing a subject's skin so as to allow drawing blood from the subject and a plurality of blood-receiving wells each of which is configured to receive a portion of the drawn blood. The dermal patch can include a plurality of blood-receiving wells each of which is configured to receive a portion of the drawn blood. In some embodiments, at least one mechanism is operably coupled to the chambers for spatially distributing the blood received in each of said chambers into a monolayer blood smear.

In some embodiments, the dermal patch can include at least a first blood-transfer fluidic channel having an inlet for receiving at least a portion of the drawn blood and an outlet through which the blood can exit the blood-transfer fluidic channel. A blood distribution fluidic device having at least one inlet in communication with the outlet of the first blood-transfer fluidic channel can receive at least a portion of the drawn blood and can include a plurality of outlets in fluid communication with the plurality of the blood receiving wells for directing, to each of the wells, a portion of the received blood. In some embodiments, the number of the blood-receiving wells can be, for example, in a range of 2 to about 20, though other number of blood-receiving wells can also be employed. Further, each of the blood-receiving wells can have a volume, for example, in a range of equal to or less than about 2 milliliters, or equal to or less than about 1 milliliter, e.g., in a range of about 10 microliters to about 1 milliliter.

In a related aspect, a dermal patch is disclosed, which includes at least a pair of sensing units each configured for detecting at least one target analyte in a physiological sample, and at least one microneedle that is configured for puncturing the skin to allow collection of the physiological sample. The dermal patch can further include a selector device for selecting any one of said sensing units for receiving at least a portion of the collected physiological sample for analysis thereof.

In some embodiments, the selector device may be implemented as any of a mechanical, electromechanical, or electromagnetic selection element.

In some embodiments, the selector device includes at least one visual indicator, e.g., a selector dial, for allowing a user to select any one of said sensing units, e.g., at different discrete times.

The dermal patch can further include a plurality of reservoirs for storing one or more reagents/buffers for processing the physiological sample to generate a processed sample, where each of said reservoirs is associated with one of said sensing units. By way of example, the processing reagents/buffers may be selected for processing of a blood sample (e.g., the reagents may include an anticoagulant, such as heparin, and/or a protease inhibitor). In some embodiments, the processing reagents (e.g., primers, etc.) are suitable for providing isothermal amplification of a target analyte (e.g., a cell free DNA segment in the blood sample). In some embodiments, the processing reagents can be in a lyophilized form when stored in the reservoir and can be reconstituted when transferred to a sample collection chamber, or before transfer. For example, the sample collection chamber may include a solvent for reconstituting the lyophilized reagents. Alternatively, such a solvent may be stored in a pouch and can be released into a reservoir in response to the selection of a sensing unit associated with that reservoir. In some embodiments, the lyophilized reagent(s) may be stored on a nitrocellulose pad.

In many embodiments, the reservoirs are pre-filled with the requisite processing reagents/buffers such that the dermal patch can be used without a need to fill the reservoirs with the processing reagents/buffers at the point of care. In other words, a user can utilize the dermal patch with all the requisite buffers and processing reagents on board. This feature provides distinct advantages in that it ensures consumer safety and reduces, and preferably eliminates, the risk of error. In other words, in many embodiments a dermal patch according to the present teachings contains all the necessary sample processing reagents/buffers for its intended use. Further, in many embodiments, the dermal patch can include the electronic circuitry that allows the processing of signals generated by one or more sensors of the dermal patch.

As discussed further below, in some embodiments, a dermal patch according to the present teachings, the processing reagent(s) and/or buffers required for detecting and optionally quantifying a target analyte may be stored in the sample collection chambers. For example, the processing reagent(s) may be stored in the sample collection chambers in a lyophilized form (e.g., on a nitrocellulose pad), or otherwise. In some such embodiments, the dermal patch does not include any reservoirs, or at least some of the sample collection chambers are not associated with a reservoir, and all the requisite processing reagent(s) and/or buffers, etc. are stored in the sample collection chambers. Alternatively, in some embodiments, all the requisite processing reagent(s)/buffers are stored in a lyophilized form in the sample collection chambers and one or more solvents required for reconstitution of the lyophilized reagent(s) may be stored in one or more reservoirs incorporated in the dermal patch (such as those disclosed herein), where in use the solvent(s) may be transferred from the reservoir(s) to the sample collection chambers. e.g., in a manner disclosed herein, for the reconstitution of the lyophilized reagent(s) stored therein.

In some embodiments, the site for application of the dermal patch is envisaged to be the forearm, or the upper arm. In some implementations, such application of the dermal patch can advantageously reduce or eliminate the need for electronics on board. In some embodiments, a wearable unit, e.g., a watch-like device, may be employed to supply power to one or more sensors of the dermal patch and/or receive signals generated by those sensor(s) for analysis and/or presentation of the analysis results to a user (the subject and/or a healthcare professional). Further, in some embodiments, the wearable device may include communication circuitry for communicating the data and/or analysis results to another device, such as an external server. By way of example, the wearable device may employ a wireless communication protocol, such as Bluetooth, Wi-Fi, etc., for communicating with the external device.

A variety of sensors may be incorporated in a dermal patch according to the present teachings. The sensors may be passive or active sensors. Some examples of sensors include, without limitation, sensors that provide chromatographic or “photo-visual,” or digital readouts. For example, such a sensor may be a colorimetric sensor, e.g., an immunoassay sensor including lateral flow sensors, as well as isothermal amplification detection systems. Some examples of other suitable sensors include, without limitation, graphene-based sensors, electrochemical sensors, and chemical sensors, among others.

Each of the sensing units may include a sample collection chamber for receiving at least a portion of said physiological sample in response to selection of the sensing unit by said selector device. In some embodiments, a sample collection chamber of a dermal patch according to the present teachings may have a volume equal to or less than about 2 milliliters, or equal to or less than about 1 milliliter, e.g., in a range of about 10 microliters to about 1 milliliter.

The dermal patch can further include at least one fluidic channel having an inlet configured to receive the physiological sample through the punctured skin and an outlet through which the received sample can be introduced into a sample collection chamber of a selected one of said sensing units.

In some embodiments, the dermal patch can include a switch for selectively establishing a fluid path between the outlet of the above fluidic channel and a selected one of the sample collection chambers. A controller can be in communication with the switch for activating the switch in accordance with a predefined temporal schedule to collect multiple physiological samples at different times.

In some embodiments, the switch may include a plurality of internal channels, where in one position of the switch one of the internal channels directs the received physiological sample into one of the sample collection chambers and in another position of the switch another one of the internal channels directs the received physiological sample into another one of the sample collection chambers. A variety of switches, such as mechanical, electromechanical and electromagnetic switches, may be employed.

In some embodiments, a fluid transfer channel is coupled to the selector device for establishing a fluid path between a selected sensing unit and a reservoir associated therewith. In some embodiments, the transfer of a processing fluid stored in a reservoir to the respective sample collection chamber may be achieved passively or actively. By way of example, in some embodiments, each of the reservoirs and a respective sample collection chamber are positioned relative to one another such that gravity can facilitate the transfer of the processing fluid stored in that reservoir to the sample collection chamber.

In some embodiments, one or more magnetic beads may be stored in at least one of the sample collection chambers, where the magnetic beads may be activated via an external magnet to cause mixing of the physiological sample and the processing fluid introduced into said at least one sample collection chamber.

Each of the sensing units can include at least one sensor that is in fluid communication with the sample collection chamber of that sensing unit, or can be brought into fluid communication with that sensing unit, for coming into contact with at least a portion of the processed sample and to generate one or more signals in response to the detection of a target analyte, when present in the sample. By way of example, the sensor can be coupled to the sample collection chamber via a sealed opening. Alternatively, the sample collection chamber may be formed of a flexible material that expands upon receiving the sample and the processing fluid so as to open a slit, thereby providing a fluid path between the sensor and the sample collection chamber. Other suitable means for interrogating a sample may also be employed. By way of example, in some cases, the interrogation of a sample may be achieved without the need for direct contact between a sensor and the sample, e.g., via optical techniques, such as fluorescent and/or Raman techniques.

In some embodiments, the dermal patch may include a circuitry that is in communication with the sensors of the dermal patch for receiving one or more signals (e.g., detection signals) generated by the sensors. The circuitry may be configured to process the signals to determine the presence of a target analyte in the sample and optionally quantify the level of the target analyte, when present in the sample.

A variety of sensors can be incorporated in a dermal patch according to the present teachings. Some examples of such sensors include, without limitation, graphene-based sensors, electrochemical sensors, colorimetric sensors, and/or optical sensors. In some embodiments, a colorimetric sensor may employ an immunoassay for the detection of the target analyte. In some embodiments in which a colorimetric sensor is employed, at least a portion of the dermal patch may include a transparent portion to allow the visualization of the sensor.

The circuitry may be implemented using the techniques known in the art as informed by the present teachings. By way of example, the circuitry may include at least one memory module for storing the signals generated by the sensors of the dermal patch. The circuitry may be configured to process the stored signals, e.g., detection signals, generated by different sensing units to determine a variation, if any, of a target analyte level at a plurality of discrete time at which the sensing units are activated. The circuitry may also include a communication module to allow communication between the circuitry and an external electronic device. Such an external electronic device may be a mobile electronic device. By way of example, in some embodiments, a variety of wireless communication protocols may be used for transmitting data from the circuitry to the external electronic device. Some examples of such wireless communication protocols may include Bluetooth, Wi-Fi, and BTLE protocol for establishing a communication link between said patch and said electronic device.

In some embodiments, the physiological sample may include any of blood and/or interstitial fluid.

In some embodiments, the target analyte may be a biomarker, e.g., a biomarker that may be indicative of a disease condition, e.g., organ damage. In some embodiments, the biomarker may be indicative of a traumatic brain injury, including a mild traumatic brain injury. Some examples of such a biomarker include, without limitation, any of myelin basic protein (MBP), ubiquitin carboxyl-terminal hydrolase isoenzyme L1 (UCHL-1), neuron-specific enolase (NSE), glial fibrillary acidic protein (GFAP), and S100-B.

In other embodiments, the dermal patch may be configured for the detection of other biomarkers, such as troponin, BNP, and HbA1C, among others.

In some embodiments, the sensing units of a dermal patch according to the present teachings may be configured to detect the same target analyte while in other embodiments the sensing units may be configured to detect different target analytes.

The number of sensing units can vary based on a particular application. By way of example, and without limitation, the number of sensing units may be in a range of 2 to about 20, e.g., 6 to 10, though other numbers may also be used.

In some embodiments, the dermal patch may include a plurality of microneedles, e.g., in a range of 10 to 20. In some embodiments, the microneedles may have a length in a range of about 100 microns to about 1500 microns in length, about 50 to about 250 microns in width, and about 1-25 microns in diameter, though other sizes may also be employed.

In some embodiments, the microneedle(s) may be movable between a retracted position and a deployed position in which the microneedle(s) are capable of puncturing the skin. The dermal patch may also include an actuation mechanism operably coupled to the microneedles for transitioning the microneedles between the retracted position and the deployed position.

In some embodiments, the dermal patch may include one or more pumps that are coupled to one or more internal fluidic channels of the dermal patch for generating a negative pressure therein in order to facilitate the flow of a sample and/or a processing fluid therethrough. In some such embodiments, the pump(s) can be positive displacement pumps.

The dermal patch may further include an adhesive layer that facilitates the attachment of the dermal patch to a patient's skin.

In a related aspect, a dermal patch is disclosed, which includes at least two sample collection chambers, each configured for receiving a physiological sample collected from a subject. The dermal patch further includes at least one reservoir for storing one or more processing reagents/buffers for processing the physiological sample so as to provide a processed sample. The dermal patch can also include at least two detection units each operably coupled to one of said sample collection chambers for detecting a target analyte, when present in the collected sample. The detection units can include one or more sensors, such as those discussed herein. The sensors may be configured to provide a quantitative level of a target analyte, when present in the sample. Alternatively, an external circuitry may be utilized to quantify the level of the target analyte based on signals generated by the sensor.

Further, the dermal patch may include a selector device for selecting any of said sample collection chambers for receiving the physiological sample. One or more microneedles may be incorporated in the dermal patch of puncturing a subject's skin to allow collecting a physiological sample.

In a related aspect, a dermal patch for counting blood cells in a sample of whole blood is disclosed, which includes a plurality of microneedles configured for puncturing a subject's skin to allow drawing a blood sample from the subject and at least one sample collection chamber for receiving at least a portion of a blood sample drawn from the subject. The dermal patch includes at least one transparent window associated with said at least one sample collection chamber to allow counting blood cells in said collected sample via acquiring an optical image of at least a portion of the collected blood sample and processing said image.

In some embodiments, the at least one sample collection chamber includes first and second sample collection chambers, where a first diluent is stored in the first sample collection chamber to facilitate obtaining a count of platelets in a blood sample received in said first sample collection chamber and a second diluent is stored in the second sample collection chamber to facilitate obtaining a count of red blood cells and white blood cells received in the second sample collection chamber.

The dermal patch can further include at least one fluidic channel for directing the blood drawn from the subject to said at least one sample collection chamber.

In some embodiments, the dermal patch can further include a microfluidic distribution device having an inlet for receiving at least a portion of the drawn blood sample and at least first and second outlets, where the first outlet is in fluid communication with the first sample collection chamber and the second outlet is in fluid communication with the second sample collection chamber. The microfluidic distribution device is configured to direct a first portion of the received blood sample to the first sample collection chamber and to direct a second portion of the received blood sample to the second sample collection chamber.

In some embodiments, the dermal patch can further include at least two additional sample collection chambers and a plasma-separation microfluidic device for separating a plasma or serum portion of at least a portion of the whole blood drawn from the subject and directing the separated plasma portion to one of said additional sample collection chambers and directing the remaining serum portion to the other of said additional sample collection chambers. In some such embodiments, the plasma-separation microfluidic device is in fluid communication with the microfluidic distribution device to receive at least a portion of the whole blood drawn from the subject. By way of example, in some such embodiments, an outlet of the microfluidic distribution device is in fluid communication with an inlet of the plasma-separation microfluidic device for transferring a portion of the whole blood drawn from the subject to the plasma-separation microfluidic device. The plasma-separation microfluidic device can include at least two outlets through one of which the separated plasma (or at least a portion thereof) is introduced into a plasma-collection chamber incorporated in the dermal patch and through the other of the two outlets the remaining serum component is introduced into a serum-collection chamber incorporated in the dermal patch.

At least one transparent window can be incorporated in the dermal patch to allow acquiring images of the plasma and the serum collected in the respective sample collection chambers. In some embodiments, the dermal patch can include multiple transparent windows, each of which is associated with one of the sample collection chambers. In other embodiments, a transparent window can be shared between at least two of the sample collection chambers.

In some embodiments, at least one sensor is coupled to at least one of the sample collection chambers, where the sensor is configured for detection of an analyte in a sample received by said at least one chamber. By way of example, at least a sensor can be coupled to the serum-collection chamber for detecting (and optionally quantifying) at least one biomarker, such as the biomarkers discussed above, in the collected serum. A variety of sensors can be employed. Some examples of such sensors include, without limitation, a graphene-based sensor, an electrochemical sensor, a colorimetric sensor, among others.

In some embodiments, the dermal patch can include a housing in which various components of the dermal patch can be disposed. In some such embodiments, the housing of the dermal patch can be formed as a single integral unit while in other embodiments the housing can be formed of separated components that can be coupled to one another. The transparent window(s) can be incorporated in the housing in register with at least one of the sample collection chambers to allow acquiring an image of a sample received in that sample collection chamber.

In some such embodiments, the microneedles can be movable between a retracted position and a deployed position. In some such embodiment, a chamber incorporated in the dermal patch can receive the microneedles in the retracted position.

In a related aspect, a system for counting blood cells in a sample of whole blood is disclosed, which includes a dermal patch for collecting a blood sample from a subject. Such a dermal patch can include at least one sample collection chamber for collecting a blood sample and at least one transparent window coupled to said sample collection chamber to allow visualization of the sample collected in the chamber.

Further understanding of various aspects of the present teachings may be obtained by reference to the following detailed description

### BRIEF DESCRIPTION OF THE DRAWINGS

FIG. 1 is a schematic view of an embodiment of a dermal patch according to the present teachings,

FIG. 2 is an exploded view of the dermal patch depicted in FIG. 1,

FIG. 3 is a schematic view of the dermal patch of FIG. 1 illustrating, among other elements, a plurality of internal channels and sample collection chambers,

FIG. 4 is a schematic front view of the dermal patch of FIG. 1,

FIG. 5 schematically depicts an implementation of a controller for use in some embodiments of a dermal patch according to the present teachings,

FIG. 6 is a schematic view of another embodiment of a dermal patch according to the present teachings in which a plurality of pumps, such as positive displacement pumps, are employed for facilitating the flow of a collected sample through various internal channels of the dermal patch,

FIG. 7A is a schematic view of another embodiment of a dermal patch according to the present teachings in which a programmable switch is incorporated for directing a sample drawn from a subject to sample collection chambers of the dermal patch in accordance with a predefined temporal schedule,

FIG. 7B schematically depicts an example of an implementation of the switch depicted in FIG. 7A,

FIG. 8 schematically depicts an example of an actuation mechanism for moving the microneedles of a dermal patch according to an embodiment of the present teachings between a retracted position and a deployed position,

FIG. 9 schematically depicts a dermal patch that includes a selector device having a visual indicator for allowing a user to select one of a plurality of sensing units for receiving a physiological sample,

FIG. 10 shows four sample collection chambers that may be incorporated into a dermal patch according to some embodiments of the present teachings,

FIG. 11 is a partial schematic view of a dermal patch according to an embodiment in which two sample collection chambers share a reservoir in which one or more sample processing reagents/buffers are stored,

FIG. 12 schematically shows that in some embodiments a dermal patch may be attached to an arm of a subject and be powered by a wearable device and/or transmit data thereto,

FIG. 13 shows schematically an inductive magnetic coupling between two coils one of which is incorporated in a dermal patch according to an embodiment and another one is incorporated in the wearable device,

FIGS. 14A-14B schematically depicts a dermal patch according to an embodiment,

FIG. 14C shows a sensor incorporated in the sample collection chamber according to an embodiment,

FIGS. 15A and 15B illustrate the structure and function of an exemplary manual pump utilizing a bulb assembly according to some embodiments,

FIGS. 16A and 16B, respectively show a needle assembly before and after punctuating the skin according to some embodiments,

FIG. 17 shows a flow chart for using a dermal patch according to some embodiments,

FIGS. 18A and 18B illustrate two different types of needle assemblies according to some embodiments,

FIG. 19 shows a cross section of another needle spring assembly according to some embodiments,

FIG. 20 shows a cantilever type needle assembly according to some embodiments,

FIG. 21 shows a high level sketch of a dermal patch according to some embodiments,

FIG. 22 shows an actuator utilizing a cam mechanism according to some embodiments,

FIGS. 23A and 23B show another actuator utilizing an annular-pinion mechanism according to some embodiments,

FIG. 24 shows yet another actuator, which utilizes rack and pinion mechanism according to some embodiments,

FIGS. 25A and 25B show yet another actuator system, which utilizes another cam mechanism according to some embodiments,

FIGS. 26A and 26B show a dermal patch assembly according to some embodiments,

FIGS. 27A and 27B depict a dermal patch utilizing a multipin actuator according to another embodiment,

FIGS. 28A and 28B depict various configurations for reagent reservoirs according to different embodiments,

FIG. 29 schematically depicts a cloud computing environment in accordance with an exemplary embodiment,

FIG. 30, schematically depicts a smart phone that is in communication with an electronic medical record (“EMR”) database and a dermal patch with a quick response (“QR” code) in accordance with an exemplary embodiment,

FIG. 31 depicts a method for updating an EMR in accordance with an exemplary embodiment,

FIG. 32 schematically depicts a dermal patch with a locking mechanism, an actuation button, and an electromechanical actuator in accordance with an exemplary embodiment,

FIG. 33 schematically depicts a dermal patch with a skin sensor in accordance with an exemplary embodiment,

FIG. 34 depicts a method for deploying microneedles of a dermal patch in accordance with an exemplary embodiment,

FIG. 35 depicts a method for unlocking a dermal patch in accordance with an exemplary embodiment,

FIG. 36 depicts another method for deploying microneedles of a dermal patch in accordance with an exemplary embodiment,

FIG. 37 depicts another method for unlocking a dermal patch in accordance with an exemplary embodiment,

FIG. 38 depicts two smart phones including a medical professional's smart phone connected to a dermal patch in accordance with an exemplary embodiment;

FIG. 39 depicts another method for deploying microneedles of a dermal patch in accordance with an exemplary embodiment,

FIG. 40 depicts another method for unlocking a dermal patch in accordance with an exemplary embodiment,

FIG. 41 depicts a metaverse network in accordance with an exemplary embodiment,

FIG. 42 depicts a computer system with a metaverse client in accordance with an exemplary embodiment, and

FIG. 43 depicts a metaverse in accordance with an exemplary embodiment.

### DETAILED DESCRIPTION

The present teachings are generally directed to dermal patches that may be utilized, for example, to measure the level of one or more target analytes in a physiological sample, e.g., a blood sample and/or an interstitial fluid. In some embodiments, a dermal patch according to the present teachings allows the collection and analysis of a plurality of physiological samples at different times, thereby facilitating the diagnosis and/or prognosis of a disease condition and/or efficacy of a therapeutic treatment. In many embodiments, a dermal patch according to the present teachings includes the requisite processing reagents/buffers (herein referred to as “processing fluid”) for processing a received sample on board. Such a feature, that is, the incorporation of the required processing reagents in the dermal patch, can provide a number of advantages, including additional safety as well as enhanced sensitivity and/or specificity. Further, in some embodiments, a dermal patch according to the present teachings allows measuring the level of a target analyte at a plurality of discrete times, thereby providing valuable information regarding the occurrence, progression, and/or amelioration of a disease condition.

Various terms are used herein in accordance with their ordinary meanings in the art, unless indicated otherwise. The term “about,” as used herein, denotes a deviation of at most 10% relative to a numerical value. The term “substantially,” as used herein, refers to a deviation, if any, of at most 10% from a complete state and/or condition. The terms “needle” and “microneedle” are used herein to broadly refer to an element that can provide a passageway, or facilitate the production of a passageway, for collecting a physiological sample, such as a blood or an interstitial fluid sample through a patient's skin, e.g., via puncturing the subject's skin. The term “transparent,” as used herein, indicates that light can substantially pass through an object (e.g., a window) to allow visualization of a material disposed behind the object. For example, in some embodiments, a transparent object allows the passage of at least 70%, or at least 80%, or at least 90%, of the visible light therethrough.

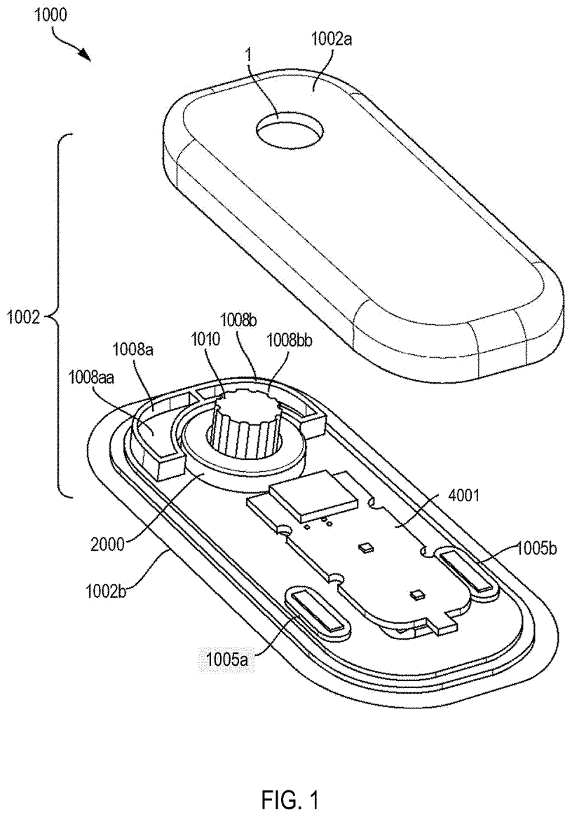

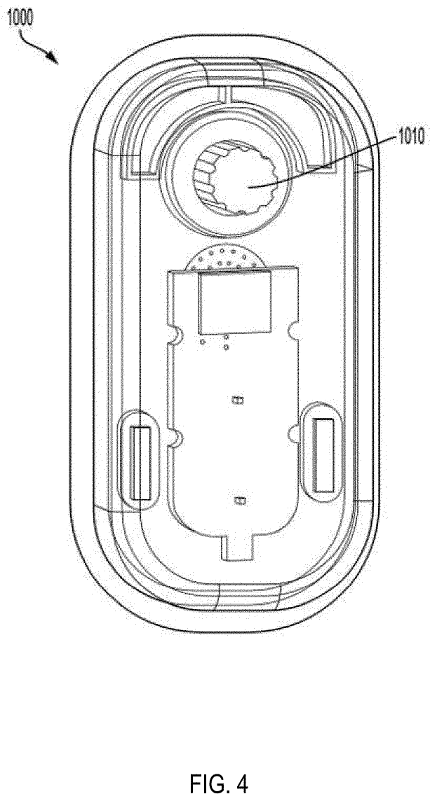

With reference to FIGS. 1, 2, 3, and 4, a dermal patch 1000 according to an embodiment of the present teachings includes a housing 1002 having a top portion 1002a and a bottom portion 1002b, which can be coupled to one another, e.g., releasably or otherwise (e.g., via glue, fasteners, etc.). While in this embodiment the housing 1002 is formed of two portions that are coupled to one another, in other embodiments the housing 1002 may be formed as a single integral unit.

The housing 1002 may be formed of any suitable polymeric material. By way of example, and without limitation, the housing 1002 may be molded from polymeric materials, such as, but not limited to, polyolefins, PET (Polyethylene Terephthalate), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, and photocrosslinkable polymers .

In some embodiments, the housing 1002 or at least a portion thereof may be formed of a transparent polymeric material, e.g. PDMS, to allow visibility of at least a portion of components disposed within the housing. By way of example, as discussed in more detail below, in some embodiments in which a colorimetric sensor is employed the transparent portion can allow visualization of a color generated by the colorimetric sensor.

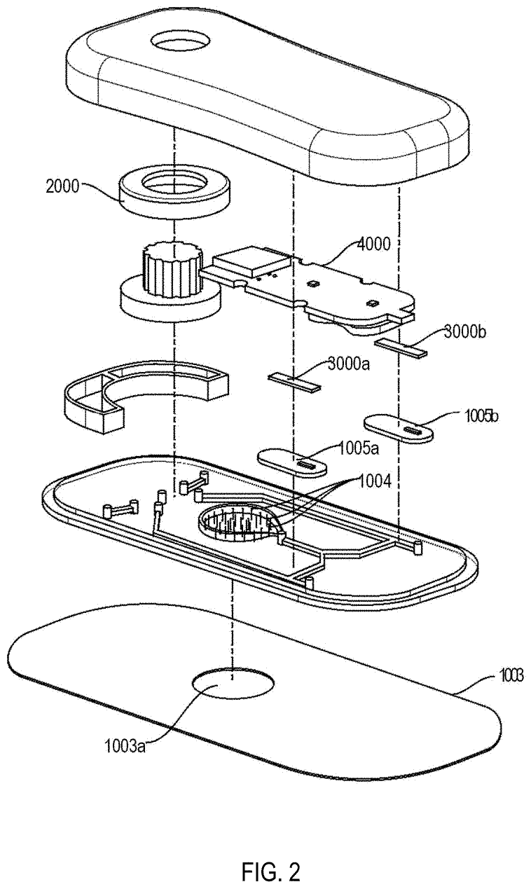

The dermal patch 1000 includes an adhesive layer 1003 that allows attaching the dermal patch to a subject's epidermal layer (See, e.g., FIG. 2).

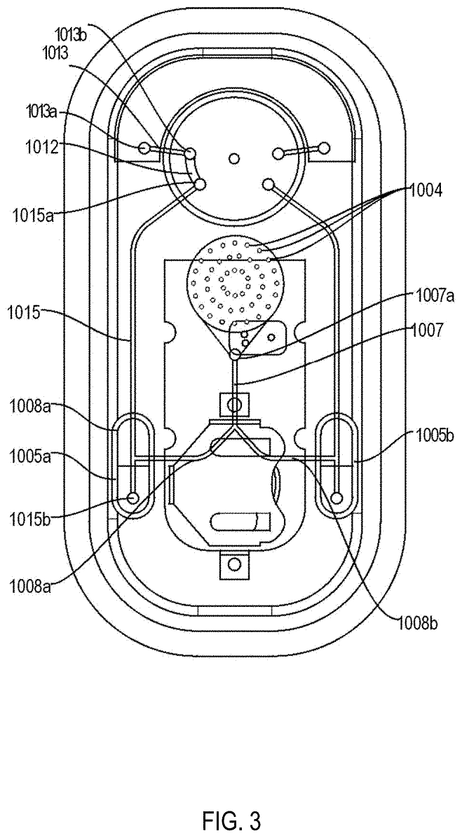

With particular reference to FIGS. 2 and 3, in this embodiment the dermal patch 1000 includes a plurality of needles 1004 (herein also referred to as microneedles) that are in register with an opening 1003a provided in the adhesive layer 1003 to allow their contact with a subject's skin upon attachment of the dermal patch to a patient's skin.

The microneedles are configured to puncture a subject's skin and penetrate through a subject's stratum corneum and at least a portion of the epidermal layer to allow collecting a physiological fluid, e.g., capillary blood and/or interstitial fluid. As discussed in more detail below, in some embodiments, the microneedles 1004 may be movable between a retracted position in which the microneedles are moved into a chamber within the dermal patch and a deployed position (herein also referred to as an extended position) in which the microneedles are exposed for puncturing the skin. In some embodiments, the microneedles 1004 may be formed of a polymeric material with a channel, e.g., a central channel, that allows collecting a physiological sample, e.g., a capillary blood sample and/or interstitial fluid. Some examples of suitable polymers include, without limitation, PDMS, epoxy siloxane polymer, among others. By way of example, in some embodiments, the needles can include an epoxy siloxane polymer layer that is sandwiched between two PDMS layers. In some cases, the polymeric needles can be fabricated using a mold.

With particular reference to FIGS. 2 and 3, in this embodiment the dermal patch 1000 includes two sample collection chambers 1005a and 1005b, each of which can receive a portion of a physiological sample drawn through the punctured skin. In particular, a fluidic channel 1007 having an inlet 1007a positioned behind the microchannels can receive the physiological sample drawn through the skin. The fluidic channel 1007 leads to two fluidic branches 1008a and 1008b, each of which is in fluidic communication with one of the sample collection chambers 1005a and 1005b to split a received physiological sample between the two sample collection chambers.

In some embodiments, the collection of the physiological sample through the punctured skin occurs passively while in other embodiments a negative pressure may be generated in the fluidic channel 1007 to facilitate the extraction of the physiological sample through the punctured skin and its transfer to the sample collection chambers.

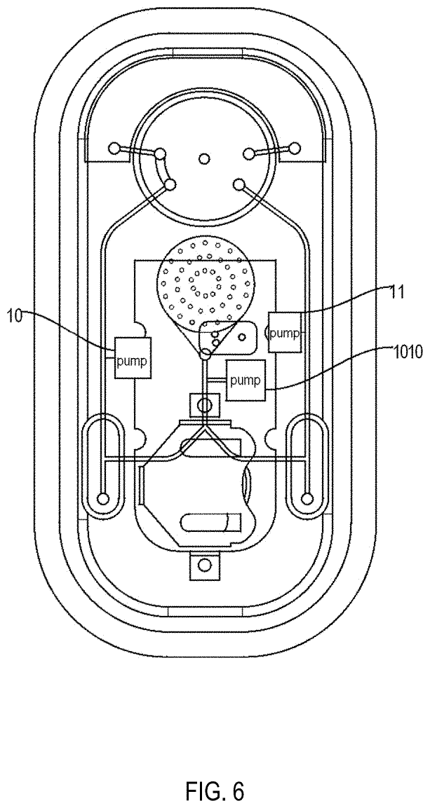

By way of example, a pump 1010 (See, e.g., FIG. 6), such as a mechanical or an electromechanical pump, may be incorporated in the dermal patch in communication with the fluidic channel 1007 to apply a negative pressure to the fluidic channel, thereby facilitating the extraction of the physiological sample through the punctured skin into the fluidic channel 1007. By way of example and without limitation, the electromechanical pump may be, e.g., a positive displacement pump.

Referring to FIGS. 1 and 2, in this embodiment, the dermal patch 1000 further includes two reservoirs 1008a and 1008b for storing one or more processing reagents/buffers (herein also referred to as a processing fluid) 1008aa/1008bb for processing the physiological samples collected within the sample collection chambers 1005a and 1005b. For example, in this embodiment and other embodiments, the processing reagents/buffers may be those that are conventionally employed during a blood analysis. In this embodiment, each of the reservoirs 1008a and 1008b is associated with one of the sample collection chambers.

A variety of processing reagents/buffers known in the art may be employed in the practice of the present teachings. By way of example, the processing fluid may be selected to facilitate the detection of a biomarker of interest. By way of example, in some embodiments, the processing fluid for processing a drawn blood sample may include an anticoagulant, such as heparin. In addition, in some cases the processing fluid may include a protease inhibitor. In yet other embodiments, the processing fluid may include reagents (such as primers, etc.) that allow isothermal amplification, e.g., for detecting cell free circulating DNA. A variety of reagents and techniques for processing of physiological samples, such as a blood sample or an interstitial fluid sample, known in the art may employed in the practice of the present teachings.

With particular reference to FIGS. 1 and 2, a selector device 1010 allows selectively connecting each of the reservoirs to its associated sample collection chamber. In this embodiment, the selector device 1010 is in the form of a selector dial that protrudes through an opening 1 provided on the top portion of the dermal patch's housing. A retaining ring 2000 facilitates the coupling of the selector device to the housing.

With particular reference to FIG. 3, in this embodiment, the selector device 1010 includes a fluidic transfer channel 1012 (e.g., a tube), which can be positioned, via rotation of the selector dial, into two positions, where in one position the fluidic transfer channel 1012 helps establish a fluidic path between one of the reservoirs 1008a/1008b and its respective sample collection chamber, and in the other position the fluidic transfer channel 1012 helps establish a fluidic path between the other reservoir and its respective sample collection chamber.

By way of example, FIG. 3 shows a position of the selector device in which the fluidic transfer channel 1012 provides a fluidic bridge between an output port 1013b of a fluidic channel 1013, which is configured to receive the processing fluid stored in the reservoir 1008a via its inlet port 1013a, and an inlet port 1015a of a fluidic channel 1015, which extends to an outlet port 1015b that is fluidically coupled to the sample collection chamber 1008a to help establish a fluidic path between the reservoir 1008a and the sample collection chamber 1005a.

In this manner, the processing fluid stored in the reservoir 1008a can be transferred to the respective sample collection chamber 1005a. In this embodiment, such transfer of the processing fluid from the reservoir 1008a to the respective sample collection chamber 1005a is facilitated by gravity. For example, a subject wearing the patch on her arm may be placed in a seated position with the arm extending downwardly so as to allow the force of gravity to help transfer at least a portion of the processing fluid stored in the chamber 1008a to the respective sample collection chamber 1005a. Alternatively, one or more pumps, such as pumps 10 and 11 shown schematically in FIG. 6 (e.g., positive displacement pumps) may be incorporated in the dermal patch to facilitate the transfer of the processing fluid from each reservoir to a respective sample collection chamber.

With particular reference to FIG. 2, in this embodiment, the dermal patch further includes two sensing units 3000a and 3000b (herein collectively referred to as the sensing units 3000) which are coupled, respectively, to the sample collection chambers 1005a and 1005b, and are configured for the detection of a target analyte in the received physiological samples. While in this embodiment the sensing units 3000 are shown as being coupled to the sample collection chambers in a horizontal direction (i.e., along a direction parallel to the outer surfaces of the top and bottom portions of the housing), in other embodiments, the sensing units 3000 may be positioned in an orthogonal (or any other suitable orientation).

In some embodiments, the volume of each of the sample collection chambers may be equal or less than about 2 milliliters, or equal to or less than about 1 milliliter, or equal to or less than about 0.5 milliliters, e.g., in a range of about 10 microliters and 1 milliliter. In many embodiments, the volume of a physiological sample, (e.g., a blood sample) drawn from a subject may be less than about 1 milliliter.

In this embodiment, each of the sensing units is in fluid communication with the respective sample collection chamber, e.g., via a sealed opening. In this embodiment, each of the sensing units includes a single sensor. In some implementations, the sensor associated with the two sample collection chambers are configured to detect the same analyte while in other implementations, the sensor associated with one sample collection chamber is configured to detect one target analyte, and the sensor associated with the other sample collection chamber is configured to detect a different analyte.

Further, in some embodiments, at least one of the sensing units may include a plurality of sensors. In some such embodiments, the plurality of sensors may be configured to detect the same target analyte, while in other embodiments the plurality of sensors may be configured to detect two or more different target analytes.

A variety of sensors may be employed in the practice of the present teachings. Some examples of suitable sensors include, without limitation, graphene-based sensors, electrochemical sensors, colorimetric sensors (e.g., sensors that employ immunoassays for the detection of a target analyte), optical sensors, among others.

In some embodiments, the sensors are configured to provide a signal indicative of the presence of a target analyte at a concentration level above a limit-of-detection (LOD) of that sensor for that target analyte. In other embodiments, the sensor may be calibrated to provide a quantitative level of the target analyte (e.g., the concentration of the target analyte in the collected sample). In addition or instead, the signals generated by a sensor may be processed via an on-board processor (as discussed further below) or an external processor to quantify the level of the target analyte detected in the sample. By way of example, such quantification may be implemented using previously-generated calibration data in a manner known in the art as informed by the present teachings.

By way of example, a target analyte (e.g., a biomarker such as those disclosed herein) may be detected via a graphene-based sensor that includes a graphene layer that is functionalized with a moiety (e.g., an antibody, an aptamer, an oligonucleotide, etc.) that exhibits specific binding to that target analyte (e.g., a protein, a DNA segment) such that upon binding of the target analyte to that moiety an electrical property of the underlying graphene layer changes, thus indicating the presence of the target analyte in the sample. Some examples of suitable graphene-based sensors are disclosed in U.S. Pat. Nos. 10,782,285, 10,401,352, 9,664,674, as well as published U.S. Patent Applications Nos. 20200011860, and 20210102937, each of which is herein incorporated by reference in their entirety.

By way of example, the detection of a target analyte may be achieved by using a graphene-based sensor and/or an electrochemical sensor that is functionalized with a probe, such as an antibody and/or aptamer, which exhibits specific binding to that target analyte, though other sensing technologies may also be utilized.

In another embodiment, the sensor can be an electrochemical sensor that can function in a faradaic or non-faradaic mode to detect a target analyte of interest. For example, such an electrochemical sensor may include a working electrode, a reference electrode and a counter electrodes. By way of example, in some embodiments, the reference electrode may be functionalized with a moiety that exhibits specific binding to a target analyte such that upon binding of that target analyte, when present in the sample, to the moiety, a change in the current through the circuit may be detected.

Other types of sensors may be employed. For example, in some embodiments, an immunosensor that employs antibodies and provide visual indication of the presence of a target analyte in a sample via a change in color may be employed. Yet, in other embodiments, optical sensors, such as fluorescent and Raman detectors, may be used.

With particular reference to FIGS. 1 and 2, the dermal patch 1000 includes a circuitry 4001, implemented on a printed circuit board (PCB), that is in communication with the sensors 3000a and 3000b such that the circuitry 4001 receives the signals generated by the sensors 3000. The connection between the circuitry 4001 and the sensors 3000 may be established via any of a wired or wireless protocol. In some implementations, the circuitry and/or the sensors 3000 can be supplied with power via an on-board power supply, e.g., a battery, incorporated, e.g., on the circuitry. Alternatively, in some implementations, the circuitry and/or the sensors can be provided with power via an external device, e.g., a wearable device. Such transfer of power from an external device may be achieved using techniques known in the art, such as inductive coupling between two elements (e.g., two coils) provided in the dermal patch and the external device.

The circuitry 4001 may be configured to process the signals generated by the sensors 3000 to determine the presence and optionally quantify the level of a target analyte in the sample. The circuitry may be implemented according to known techniques in the art as informed by the present teachings. For example, the circuitry may include an ASIC that is configured for processing the signal data generated by the sensors. The circuitry can further include one or more memory modules for storing, for example, instructions for processing the data generated by the sensors. In some embodiments, the circuitry 4001 may transmit data (e.g., data related to the level (e.g., concentration) of a target analyte in a physiological sample) to an external device, such as a mobile phone, a server, for example, for presentation to a user (e.g., a patient and/or a healthcare professional), for further analysis and/or storage. In some embodiments, the circuity is configured to communicate the data via a wireless protocol, such as Bluetooth, Wi-Fi, and BTLE protocol, though any other suitable protocol may also be employed.

In the above embodiments, the physiological sample is drawn into the sample collection chambers, and the selector device 1010 is used for selective fluidic coupling of one of the reservoirs to a respective sample collection chamber. In other words, in the above embodiment, while both sample collection chambers receive portions of the sample substantially concurrently, the interrogation of the sample portions received in the two sample collection chambers can be done at different times.

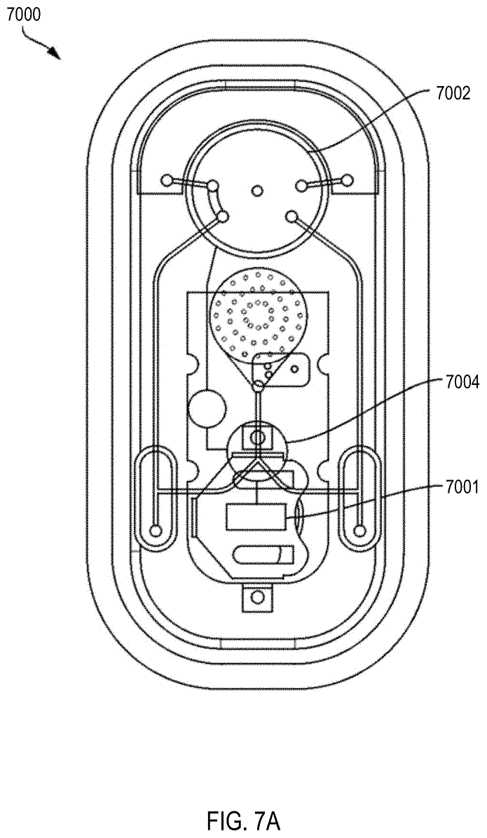

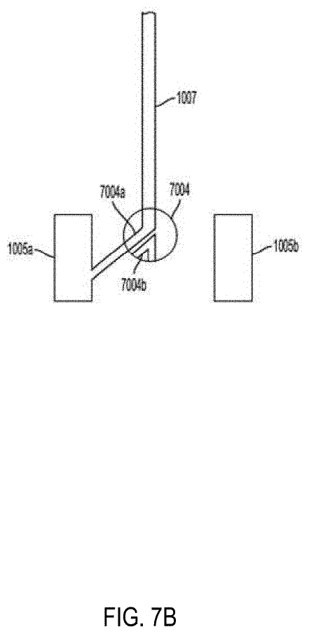

In other embodiments, the dermal patch may be configured such that different samples can be collected into the two sample collection chambers at different times. By way of example, the selector device can activate a switch to direct a physiological sample drawn through the punctured skin into one or the other of the two sample collection chambers. By way of example, FIG. 7A schematically depicts a dermal patch 7000 according to such an embodiment in which a selector device 7002 is configured to activate a switch 7004 in response to the selection of one of two sample collection chambers 7006a/7006b, e.g., via a relay 7005, to direct a physiological sample drawn through a subject's punctured skin into the selected sample collection chamber.

By way of example, with reference to FIG. 7B, in this embodiment, the switch 7004 includes internal channels 7004a/7004b, which can be selectively coupled to the outlet of the fluidic channel 1007, e.g., via rotation of a platform on which the fluidic channels are disposed, so as to direct a physiological sample flowing through the fluidic channel 1007 into the sample collection chamber 1005a or the sample collection chamber 1005b.

Further, in response to the selection of one of the sample collection chambers via the selector device, the selector device connects a reservoir containing a processing fluid that is associated with that sample collection chamber to the chamber, e.g., in a manner discussed above.

Similar to the previous embodiments, one or more fluidic channels within the dermal patch may be evacuated to a pressure below the atmospheric pressure so as to facilitate the flow of various fluids (e.g., the physiological sample and/or the processing fluid) through those channels.

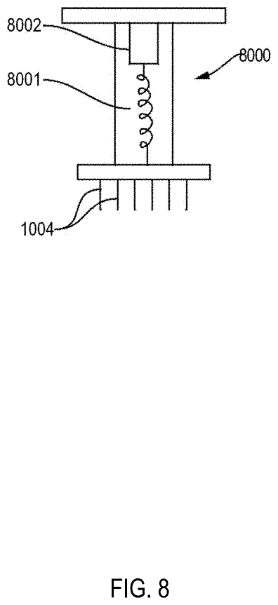

In some embodiments of any of the above dermal patches, the microneedles can be transitioned from a retracted position to a deployed position for puncturing the skin. By way of example, with reference to FIG. 8, in some such embodiments, an actuation mechanism 8000 can move the microneedles between the retracted and the deployed position. The actuation mechanism can be activated, for example, mechanically or electromechanically. For example, in this embodiment, a spring 8001 that can be transitioned between a compressed and an extended position via rotation of a knob 8002, which can in turn cause the linear motion of a piston 8003 coupled to one end of the spring so as to move the spring between a compressed and an extended state so as to transition the microneedles 1004 between a retracted and a deployed state, respectively.

In some embodiments, a dermal patch according to the present teachings may not include any reservoirs and the processing reagent(s) needed for processing a collected sample may be stored within the sample collection chambers. By way of example, such a dermal patch may be similar to that shown in FIG. 1 but without the reservoirs and their associated fluidic channels. In some implementations of such a dermal patch, the processing fluid may be stored in a sample collection chamber in a lyophilized form, and can be reconstituted upon the introduction of a collected sample into the sample collection chambers. Further, in some embodiments, some of the sample collection chambers may contain the requisite sample processing reagent(s) while other sample collection chambers may be coupled to reservoirs for receiving the processing reagent(s).

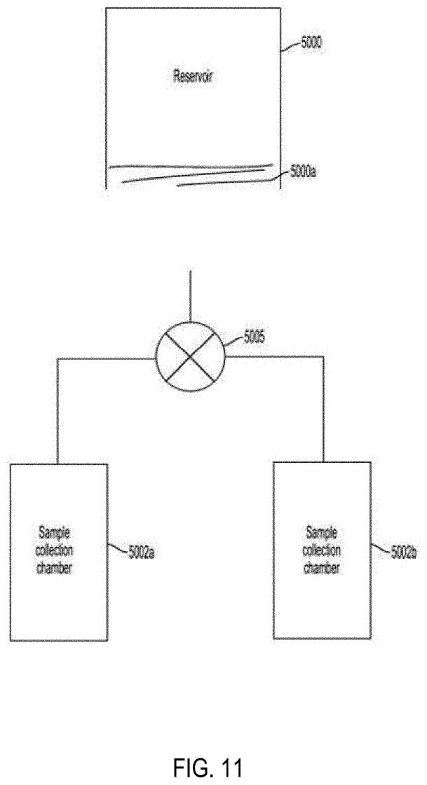

Further, in some embodiments, a dermal patch according to the present teachings may be configured such that two or more of the sample collection chambers share a reservoir. By way of example, FIG. 11 is a partial schematic view of such a dermal patch, which includes a single reservoir 5000 containing processing reagents 5000a, which is shared between two sample collection chambers 5002a/5002b. A switch 5005 can be activated to direct the processing reagent(s) stored in the reservoir into one or the other of the sample collection chambers.

A dermal patch according to the present teachings, such as the above dermal patches, may be employed to detect a variety of physiological target analytes in a sample drawn from a subject, e.g., a blood sample and/or an interstitial fluid sample, including a variety of biomarkers. Some examples of such target analytes include, without limitation, Cardiac troponin I protein (cTnI), Cardiac troponin T protein (cTnT), C-reactive protein (CRP), B-type natriuretic peptide (BNP), Myeloperoxidase, Creatine kinase MB, Myoglobin, Hemoglobin, HbA1C.

Further, in some embodiments, the dermal patch may be configured to detect one or more biomarkers for diagnosis of brain damage, such as traumatic brain injury (TBI). Some examples of such biomarkers include, without limitation, myelin basic protein (MBP), ubiquitin carboxyl-terminal hydrolase isoenzyme L1 (UCHL-1), neuron-specific enolase (NSE), glial fibrillary acidic protein (GFAP), and S100-B.

By way of example, the dermal patch may be configured to measure levels of the protein biomarkers UCHL-1 and GFAP, which are released from the brain into blood within 12 hours of head injury. The levels of these two proteins measured by a dermal patch according to the present teachings after a mild TBI can help identify those patients that may have intracranial lesions. In some such implementations of a dermal patch according to the present teachings, each sensing unit associated with a sample collection chamber can have at least two sensors one of which is configured for the detection of UCHL-1 and the other for the detection of GFAP. In addition or alternatively, the dermal patch may include one set of sample collection chambers dedicated to the detection UCHL-1 and another set of sample collection chambers that are dedicated to the detection of GFAP. The dermal patch may then be employed to obtain the levels of these proteins in blood samples drawn from a patient at different times, thereby facilitating the diagnosis of TBI.

In some embodiments, a dermal patch according to the present teachings, such as those discussed above, may include a controller that can be programmed to cause the collection of a sample (e.g., a blood sample) according to a predefined temporal schedule. By way of example, with reference to FIGS. 7A and 7B, in some implementations of the above dermal patch 7000, the on-board circuitry may include a computer system 7001 that may be programmed to activate the selector device 7002 so as to allow a physiological sample, e.g., a blood sample or an interstitial fluid sample, to be drawn into the sample collection chambers 7004a and 7004b at different times for analysis.

The computer system 7001 may be implemented in any of hardware, software and/or firmware in a manner known in the art as informed by the present teachings.

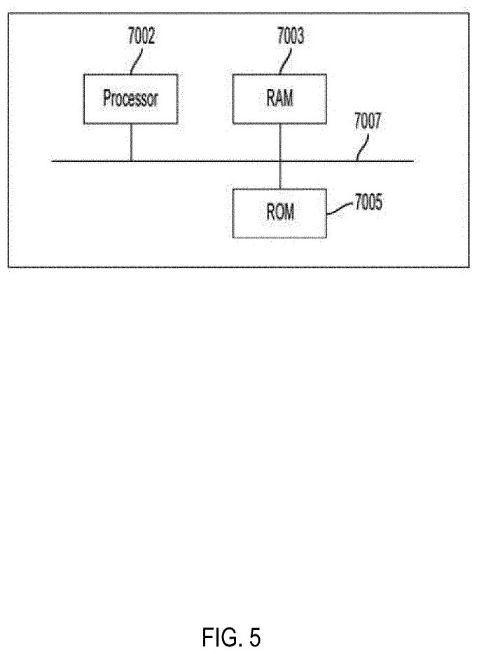

Referring now to FIG. 5, the computer system 7001 is shown in accordance with an exemplary embodiment. As used herein a computer system (or device) is any system/device capable of receiving, processing, and/or sending data. Computer systems include, but are not limited to, microprocessor-based systems, personal computers, servers, hand-held computing devices, tablets, smartphones, multiprocessor-based systems, mainframe computer systems, virtual reality (“VR”) headsets and the like.

As shown in FIG. 5, the computer system 7001 includes one or more processors or processing units 7008, a system memory 7010, and a bus 7012 that couples various components of the computer system 7001 including the system memory 7010 to the processor 7008.

The system memory 7010 includes a computer readable storage medium 7014 and volatile memory 7016 (e.g., Random Access Memory, cache, etc.). As used herein, a computer readable storage medium includes any media that is capable of storing computer readable program instructions and is accessible by a computer system. The computer readable storage medium 7014 includes non-volatile and non-transitory storage media (e.g., flash memory, read only memory (ROM), hard disk drives, etc.). Computer readable program instructions as described herein include program modules (e.g., routines, programs, objects, components, logic, data structures, etc.) that are executable by a processor. Furthermore, computer readable program instructions, when executed by a processor, can direct a computer system (e.g., the computer system 7001) to function in a particular manner such that a computer readable storage medium (e.g., the computer readable storage medium 7014) comprises an article of manufacture. Specifically, when the computer readable program instructions stored in the computer readable storage medium 7014 are executed by the processor 7008 they create means for activating the switch 7004 according to a predefined temporal schedule, e.g., for collecting a physiological sample may be stored in the computer readable storage medium 7014, e.g., at times separated from one another by one hour.

The bus 7012 may be one or more of any type of bus structure capable of transmitting data between components of the computer system 7001 (e.g., a memory bus, a memory controller, a peripheral bus, an accelerated graphics port, etc.).

The computer system 7001 may further include a communication adapter 7018 which allows the computer system 7001 to communicate with one or more other computer systems/devices via one or communication protocols (e.g., Wi-Fi, BTLE, etc.) and in some embodiments may allow the computer system 7001 to communicate with one or more other computer systems/devices over one or more networks (e.g., a local area network (LAN), a wide area network (WAN), a public network (the Internet), etc.).

In some embodiments, the computer system 7001 may be connected to one or more external devices 7020 and a display 7022. As used herein, an external device includes any device that allows a user to interact with a computer system (e.g., mouse, keyboard, touch screen, etc.). An external device 7020 and the display 7022 may be in communication with the processor 7008 and the system memory 7010 via an Input/Output (I/O) interface 7024.

The display 7022 may display a graphical user interface (GUI) that may include a plurality of selectable icons and/or editable fields. A user may use an external device 7020 (e.g., a mouse) to select one or more icons and/or edit one or more editable fields. Selecting an icon and/or editing a field may cause the processor 7008 to execute computer readable program instructions stored in the computer readable storage medium 7014. In one example, a user may use an external device 7020 to interact with the computer system 7001 and cause the processor 7008 to execute computer readable program instructions relating to at least a portion of steps of the methods disclosed herein.

While FIG. 7A depicts the dermal patch 7000 as including the computer system 7001, in some embodiments, the computer system 7001 may be omitted.

By way of example, such a dermal patch would allow monitoring one or more biomarkers at different discrete times. Such monitoring of a biomarker level at different discrete times may be employed, for example, in the diagnosis of a disease condition and/or the progression of a disease condition, and/or the response of a patient to a therapeutic regimen. By way of example, when a subject is suspected to have suffered from a traumatic brain injury (e.g., concussion), the monitoring of a TBI-related biomarker at a plurality of discrete times may help with diagnosis of TBI and its temporal progression.

In another application, a dermal patch according to the present teachings can be used to assess the progression of organ damage. By way of example, the dermal patch may be used to monitor the level of troponin in a cardiac patient at a plurality of discrete times to assess the temporal progression of damage to the patient's heart muscle tissue.

In other embodiments, rather than employing a pre-programmed schedule of sample collection, the patch can allow, e.g., via the activation of the selector device by a user (e.g., a patient and/or a healthcare provider), to collect a plurality of physiological samples (e.g., blood samples) from the patient at different times based on the user's decision.

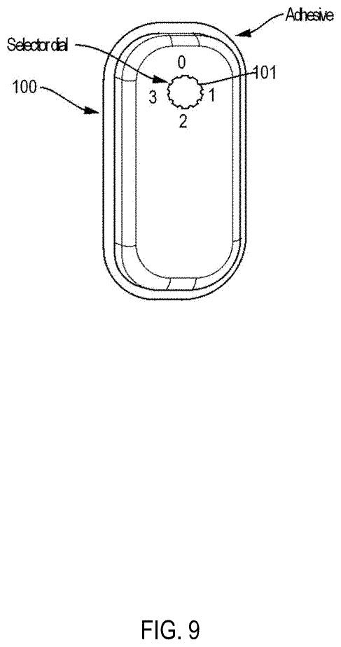

In many embodiments, the selector device of a dermal patch according to the present teachings may include a visual indicator that allows a user to select one of the sample collection chambers for receiving a physiological sample and/or a sample processing fluid. By way of example, as shown schematically in FIG. 9, a dermal patch 100 according to the present teachings includes a selector dial 101 having a visual indicator in the form of an arrow that can be aligned with each of a plurality of reference numerals, each associated with one of the sample collection chambers, to select the sample collection chamber associated with the selected reference numeral.

In some embodiments, a dermal patch according to the present teachings may include an indicator that shows which of the sample collection chambers have already been used for collecting the physiological sample. By way of example, such an indicator may be a light indicator that changes color, e.g., from green to red, to indicate that a sample collection chamber contains a sample.

Although in the above embodiments, the dermal patches are depicted to include two sample collection chambers, the present teachings are not restricted to dermal patches having only two sample collection chambers. For example, in other embodiments, four or more (e.g., up to 10), sample collection chambers may be employed.

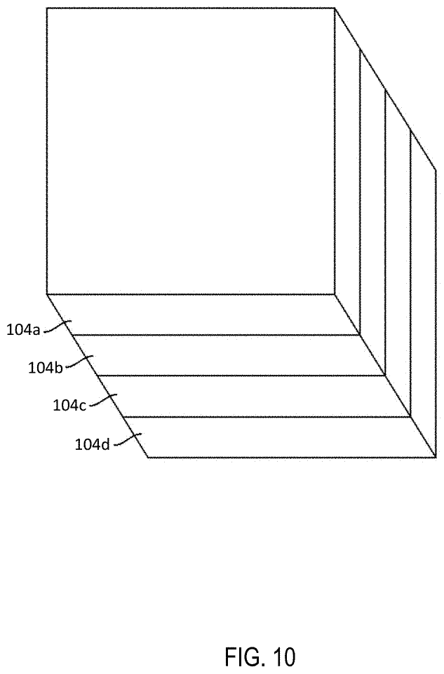

By way of illustration, with reference to FIGS. 9 and 10, the dermal patch 100 includes four sample collection chambers 104a, 104b, 104c, 104d, which are configured for collection of a physiological sample. In this embodiment, the sample collection chambers are in the form of parallelopipeds, though in other embodiments other shapes may be used. Further, while in this embodiment the sample collection chambers are stacked next to each other along the horizontal direction, i.e., with their long dimensions perpendicular to the top and bottom surfaces of the patch, in other embodiments, the sample collection chambers may be stacked along an orthogonal direction (herein also referred to as the “vertical direction”), i.e., with their long dimensions parallel to the two opposed surfaces of the patch, or may be stacked relative to one another in any other suitable configuration. Similar to the previous embodiments, each of the sample collection chambers may be associated with a respective reservoir in which a processing fluid may be stored.

In some embodiments, a dermal patch, such as those disclosed above, does not incorporate an electronic circuity and/or a power supply. For example, in some such embodiments, a colorimetric sensor, such as an immunoassay sensor (e.g., a lateral flow immunosensor) with or without isothermal amplification of a target analyte, may be incorporated into the dermal patch to allow detecting (and optionally quantifying) a target analyte without a need to supply power to the detector. The output of such a sensor can be observed chromatographically and/or via “photo-visual” read.

Alternatively, a dermal patch having a sensor that requires power may be energized externally. For example, such a sensor may receive power via a mobile device, such as a wearable device. In other words, in some embodiments, the dermal patch together with a wearable device that can supply power to the dermal patch provide a modular system. By way of example, such transfer of power from an external source to the sensor can be achieved via an inductive coupling between the sensor and the external power source.

A dermal patch according to the present teachings can be attached to any suitable site of a subject's body. By way of example, the dermal patch may be attached to a subject's arm, e.g., a forearm or an upper arm. The attachment of the dermal patch to a subject's arm can be particularly convenient in cases when a wearable device may be employed for supplying power to the dermal patch, e.g., in a manner discussed above. In some such embodiments, the sensor can include a communication module that allows transmitting signals generated by the sensor to an external device, e.g., a wearable device being worn by the user, for analysis and presentation.

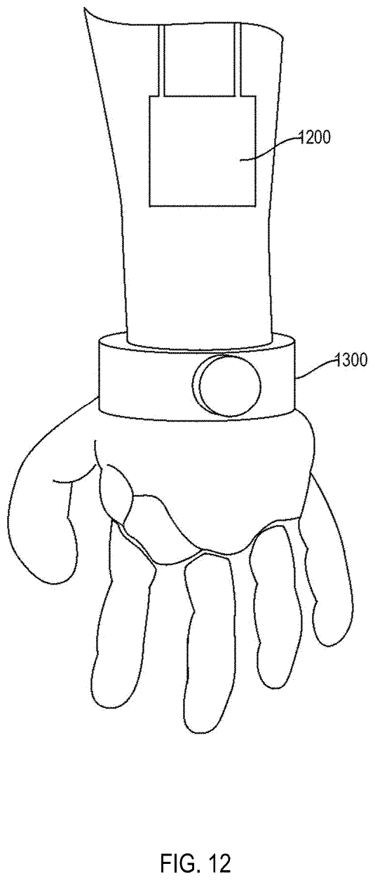

For example, as shown schematically in FIG. 12, in some embodiments, the circuitry and/or the sensors associated with a dermal patch 1200 according to the present teachings may be supplied with power via a wearable device 1300 worn by a subject. Although FIG. 12 shows the dermal patch and the wearable device being worn on the same arm, it may be more convenient for the subject to wear the dermal patch on one arm and the wearable device on the other arm so as to allow the wearable device to be brought into close proximity, and/or in contact, with the dermal patch for supplying power thereto. Alternatively, the dermal patch and the wearable device may be worn on the same arm in sufficient proximity to one another to allow transfer of power and/or data therebetween.

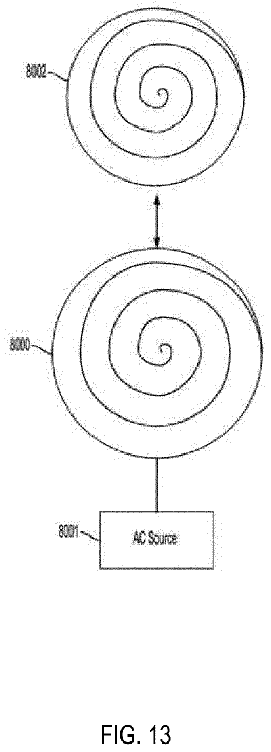

In some embodiments, an inductive coupling between the wearable device and the dermal patch may be employed for transferring power from the wearable device (or another external device) to the dermal patch. For example, as shown schematically in FIG. 13, the wearable device may include a coil (or a stack of coils) 8000 that may be energized via a power source 8001 (typically an AC power source) to generate an oscillating magnetic field, which can be inductively coupled to a respective coil 8002 incorporated in the dermal patch to generate a current in the coil 8002. Any other suitable means for transferring power from the wearable device to the dermal patch may also be employed. In some embodiments, the teachings disclosed in “Wireless Technologies for Implantable Device,” published on Aug. 16, 2020 online in PubMed (PMID: 32824365), which is incorporated herein by reference in its entirety, as informed by the present teachings, may be employed for communicating and/or supplying power to a dermal patch according to the present teachings.

Further, as noted above, in some embodiments, a dermal patch according to the present teachings may include communication circuitry, such as Bluetooth, for transmitting data (e.g., signals generated by the sensors) to an external device, such as the wearable device discussed above.

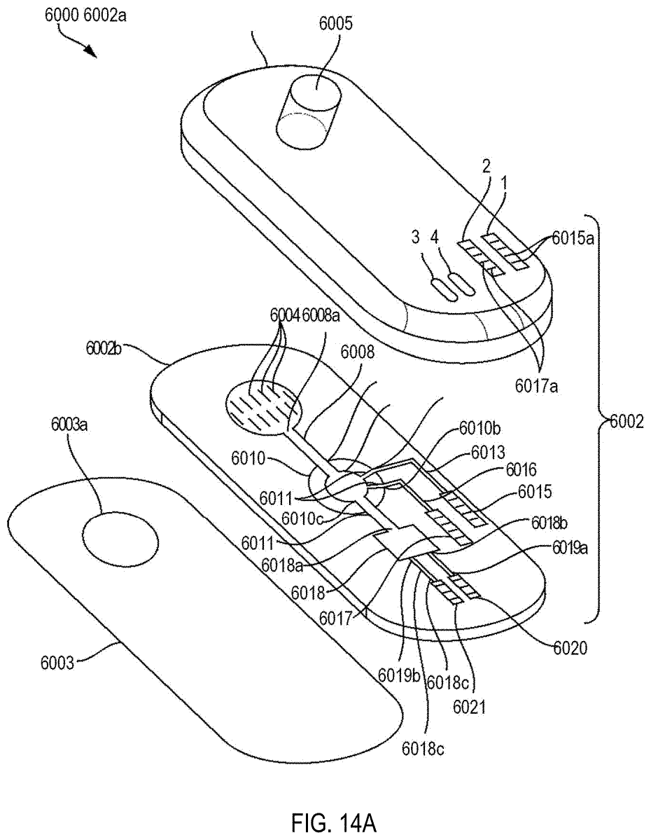

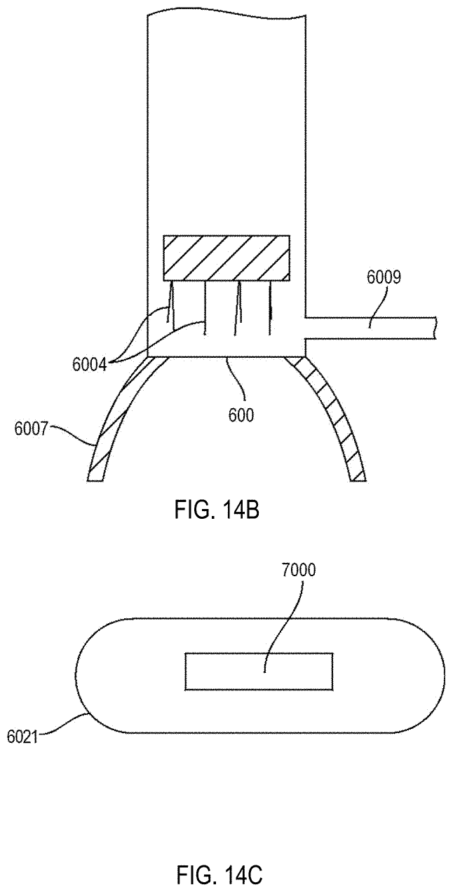

FIG. 14A schematically depicts a dermal patch 6000 according to an embodiment, which similar to the above embodiments includes a housing 6002 having a top portion 6002a and a bottom portion 6002b, which can be coupled to one another, e.g., releasably or otherwise (e.g., via glue, fasteners, etc.). While in this embodiment the housing 6002 is formed of two portions that are coupled to one another, in other embodiments the housing 6002 may be formed as a single integral unit.

Similar to the above embodiments, the housing 6002 may be formed of any suitable polymeric material. By way of example, and without limitation, the housing 6002 may be molded from polymeric materials, such as, but not limited to, polyolefins, PET (Polyethylene Terephthalate), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, and photocrosslinkable polymers.

The dermal patch 6000 includes an adhesive layer 6003 that allows attaching the dermal patch to a subject's skin surface.

Similar to the previous embodiments, the dermal patch 6000 includes a plurality of needles 6004 (herein also referred to as microneedles) that are in register with an opening 6003a provided in the adhesive layer 6003. As discussed further below, the microneedles can be transitioned between a retracted position and a deployed position.

The microneedles are configured to puncture a subject's skin and penetrate through a subject's stratum corneum and at least a portion of the epidermal layer to allow collecting capillary blood. As discussed above, and shown schematically in FIG. 14B, in some embodiments, the microneedles 6004 may be movable between a retracted position in which the microneedles are moved into a chamber within the dermal patch and a deployed position (herein also referred to as an extended position) in which the microneedles are exposed for puncturing the skin.

With reference to FIGS. 14A and 14B, in use, the dermal patch can be attached to a subject's skin via the adhesive layer 6003. The microneedles can be actuated via a button 6005 so as to be moved from a retracted position into a deployed position to puncture the skin and allow drawing blood into the dermal patch. More specifically, as shown in FIG. 14B, the dermal patch 6000 can include a cavity 6007 through which the microneedles can extend for puncturing the skin. In this embodiment, a frangible sealing membrane 600 covers an opening of the cavity such that when the microneedles are transitioned from the retracted position into the deployed position, the microneedles puncture the sealing membrane. Once the microneedles are retracted, the blood can flow through a lumen 6009 having an inlet in the cavity into a fluidic channel 6008 (See, FIG. 14A) via an inlet 6008a thereof. In some embodiments, the lumen 6009 can be maintained at a negative pressure (e.g., passively or actively), so as to facilitate the extraction of blood (e.g., capillary blood) into the lumen. For example, in some such embodiments, the dermal patch may include an evacuated chamber separated from the lumen 6009 via a membrane that can be punctured as the microneedles move from the retracted position to the deployed position so as to equilibrate the pressure between the lumen and the evacuated chamber, thereby generating a negative pressure within the lumen.

With particular reference to FIG. 14A, the fluidic channel 6008 extends from the inlet 6008a to an outlet 6008b that is in fluidic communication with an inlet of a fluidic distribution device 6010. In this embodiment, the fluidic distribution device 6010 includes three outlets 6010a, 6010b, and 6010c through each of which a portion of the blood sample received via the inlet of the fluidic distribution device can exit the fluidic device.

More specifically, in this embodiment, the fluidic distribution device 6010 includes a plurality of internal fluidic channels 6011 for directing different portions of the received sample to the outlets 6010a, 6010b, and 6010c. Further, in this embodiment, the fluidic distribution device 6010 can include a microfluidic pump 6012 (e.g., a positive displacement pump), which can facilitate the distribution of the received blood to the outlets 6010a, 6010b, and 6010c.

In other embodiments, the fluidic distribution device 6010 can function without a pump (i.e., as a passive device). While in this embodiment each of the outlets 6010a, 6010b, and 6010c receives substantially the same volume fraction of the received blood, in other embodiments, the fluid distribution device 6012 can be configured such that the volume fraction of the blood distributed to the different outlets be different.

The blood portion exiting the fluidic distribution device via the outlet 6010a flows via a fluidic channel 6013 into a sample collection chamber 6015 while the blood portion exiting via the outlet 6010b flows through a fluidic channel 6016 into another sample collection chamber 6017. In this embodiment, the sample collection chamber 6015 contains a diluent (not shown) that is suitable for processing the blood sample received in the sample collection chamber 6015 for facilitating the counting of platelets in that blood sample. A variety of diluents known in the art for processing a blood sample may be employed.

Further, the sample collection chamber 6017 contains another diluent (not shown) that is suitable for processing the blood sample received in that sample collection chamber for facilitating the counting of red and white blood cells. In some embodiments, each of the sample collection chambers 6015 and 6017 can further contain a quantity of an anti-coagulant reagent (e.g., heparin) to inhibit coagulation of a blood sample received by that sample collection chamber.

With continued reference to FIG. 14A, in this embodiment, the dermal patch 6000 includes two transparent windows 1 and 2 provided on the upper portion of the housing that are in register with the sample collection chambers 6105 and 6017. Further, at least a top surface of each of the sample collection chambers 6105 and 6017 is also formed of a transparent polymeric material to allow visualization of the blood sample collected in those chambers. In some embodiments, the volume of each of the sample collection chambers can be in a range of about 10 microliters to about 1 milliliter, though other volumes may also be employed. A plurality of graduated markings 6015a/6017a provided on the transparent windows of the sample collection chambers allows a user to determine a volume of sample received in that sample collection chamber. In some cases, a single marking can be employed to indicate the introduction of a predefined volume of a blood sample into the chamber (e.g., when the level of the blood sample received in the chamber reaches that marking).

Referring again to FIG. 14A, in this embodiment, a portion of the blood sample that exits the blood distribution device 6010 via its outlet 6010c is received by a plasma separation microfluidic device 6018 that is configured to separate plasma from whole blood (e.g., separating the plasma from whole human blood).

In this embodiment, the plasma separation microfluidic device 6018 includes an inlet 6018a for receiving a blood sample that exits via the outlet 6010c of the fluidic distribution device 6010 and further includes two outlets 6018b and 6018c, where the outlet 6018b is coupled via a fluidic channel 6019a to a sample collection chamber 6020 and the outlet 6018c is coupled via another fluidic channel 6019b to another sample collection chamber 6021. In this embodiment, the plasma separated from the whole blood exits the microfluidic separation device 6010 via its outlet 6018b to be received by the sample collection chamber 6020, and the serum component exits the microfluidic separation device 6010 to be received by the sample collection chamber 6021.

In some implementations of such a microfluidic plasma separation device, a combination of biophysical and geometrical effects may be employed for effecting the separation of plasma from whole blood. An example of such a microfluidic device that may be incorporated into an embodiment of a dermal patch according to the present teachings is disclosed in an article entitled “Microdevice for plasma separation from whole human blood using biophysical and geometrical effects,” which is herein incorporated by reference and a copy of which is reproduced as Appendix A.

Briefly, this plasma separation microfluidic device includes a main channel for blood inlet, a main channel for blood outlet and a plasma channel. The microfluidic device further includes a zone of constriction-expansion, which facilitates the separation of plasma from whole blood. More particularly, this device employs Zweifac-Fung bifurcation law by manipulating the flow rate ratio at a bifurcation. The device further includes a bent microchannel in which the cells experience a centrifugal force that pushes the cells toward the outer wall of the microchannel. The article indicates that an expansion zone positioned immediately at the end of the bent microchannel results in a significant increase of the cell free region in this zone. Hence, the device disclosed in this article employs a hybrid combination of constriction-expansion and bends for separating plasma from whole blood.

A plurality of transparent windows 3, 4 provided on the upper portion of the housing. that are in register with the collection chamber 6020 and 6021 allow interrogating the samples collected in these chambers, e.g., via capturing images of the samples. Further, one or more markings can be provided on such windows to allow obtaining an indication of the volume of a sample received in the sample collection chamber.

In some embodiments, one or more sensors can also be incorporated in the dermal patch in communication with any of the sample collection chambers 6015, 6017, 6020, and 6021 for detecting one or more analytes of interest in the samples collected in those chambers. One or more of the sample collection chambers may be coupled to such sensors. A variety of sensors may be employed. By way of example, similar to the previous embodiments, the sensor can be any of an electrochemical sensor, a graphene-based sensor, a colorimetric sensor (such as those that employ immunoassay techniques for detection of analytes), among others.

By way of illustration, FIG. 14C shows a sensor 700 incorporated in the sample collection chamber 6021 for measuring, e.g., one or more biomarkers present in the blood serum collected in that chamber. Similarly, one or more sensors can be coupled (incorporated) in one or more other sample collection chambers to measure one or more analytes (e.g., biomarkers) in the samples collected in those chambers.

Various embodiments may utilize different mechanisms for generating a vacuum and for collecting the blood sample. Some embodiments, for example, may use an active mechanism such as an automatic pump or a manual pump, or passive mechanisms such as the pull up gravity or capillary forces to drive the blood into blood processing areas such as collection chambers discussed above.

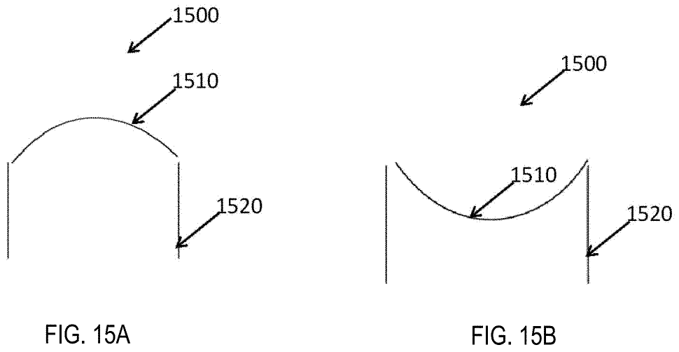

FIGS. 15A and 15B illustrate the structure and function of an exemplary manual pump utilizing a bulb assembly 1500 according to some embodiments. Bulb assembly 1500 includes a bulb 1510 and a tube 1520. The bulb 1510 may cover an end of the tube 1520. Through its other end, the tube 1520 may be connected to the end of a channel through which the blood may flow after the skin is punctured. In its initial state, the bulb 1510 may be in a convex form when viewed from outside, as shown in FIG. 15A. In order to generate a vacuum, an operator may depress the bulb 1510 into the tube 1520, as shown in FIG. 15B. After the operator releases the depressed bulb 1510, the bulb may move back towards its initial convex form, therefore lowering the pressure inside the tube 1520.

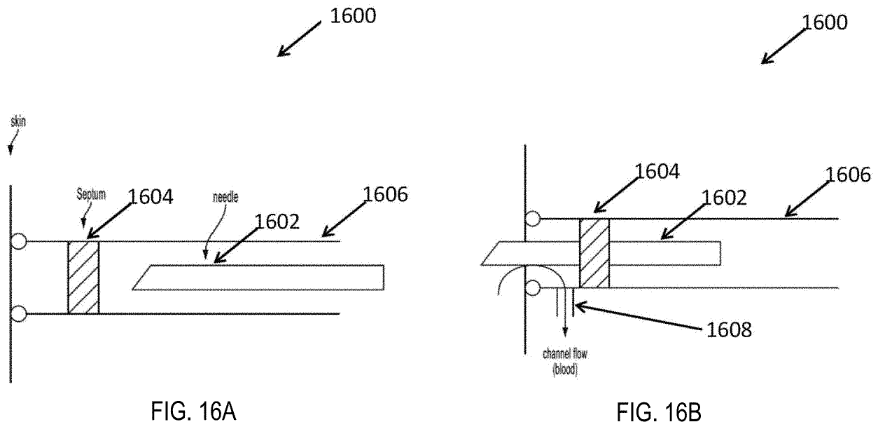

Different embodiments may use different mechanisms for puncturing the skin and directing the blood to the channel. FIGS. 16A and 16B, respectively show a needle assembly 1600 before and after punctuating the skin according to some embodiments. Needle assembly 1600 includes a needle 1602, a septum 1604, and a wall 1606. In some embodiments, the wall 1606 forms a tubular container (herein also referred to as an enclosure) for needle 1602. Some embodiments such as the needle assembly 1600 shown in FIG. 16B, further include a flow channel 1608. The needle 1602 can be moved between a retracted position in which the needle is positioned behind the septum in the tubular container, and a deployed position in which the needle has pierced through the septum and at least its tip is exposed and is available for puncturing the skin.

As shown in FIG. 16B, in order to puncture the skin, the needle 1602 passes through the septum 1604 and then punctures the skin as shown. After the skin is punctured, and before or after the needle 1602 is retracted, the blood may flow out of the punctured skin to be collected. In some embodiments, such as the needle assembly 1600 shown in FIG. 16A, the blood may be collected through the tubular wall 1606. Alternatively, and in some embodiments, such as the needle assembly 1600 in FIG. 16B, the blood may be collected through a flow channel 1608.

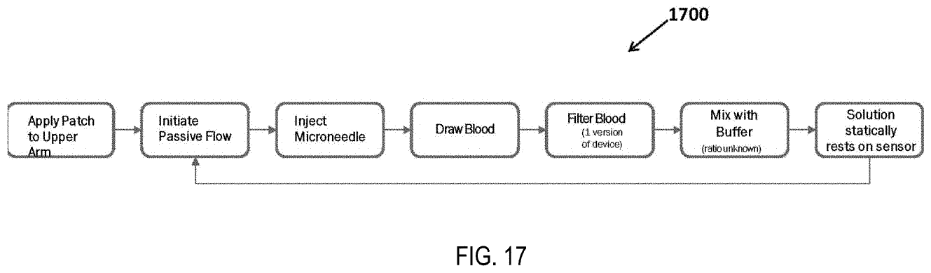

FIG. 17 shows a flow chart 1700 for using a dermal patch according to some embodiments. More specifically, flowchart 1700 starts with the step of applying the patch to the upper arm. In the next step, a flow of the blood is initiated. In particular, in the next step the patch injects one or more microneedles into the skin. In the next step, the patch draws blood from the subject. Next, the patch filters the blood. Next, the patch mixes the blood with the buffer. In some embodiments, before the mixing, the patch separates the serum from the blood cells. Next, the mixture rest statically on the sensor and the relevant data are derived.

Various embodiments utilize different mechanisms for engaging the needle and puncturing the skin. Some embodiments, for example, use different configurations of needles and springs.

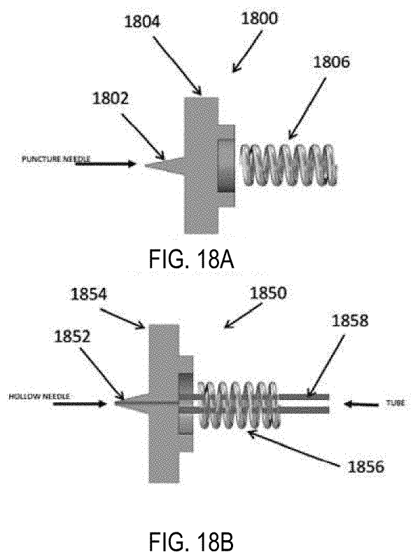

By way of example, FIGS. 18A and 18B illustrate two different types of needle assemblies according to some embodiments. In particular, FIG. 18A shows a needle spring assembly 1800, which includes a needle tip 1802, a needle cap 1804, and a spring 1806. In assembly 1800, needle tip 1802 is a solid puncture needle. In some embodiments, the puncture needle is used to puncture the skin and then withdrawn, allowing the blood to flow out of the puncture location.

FIG. 18B, on the other hand, shows a different needle spring assembly 1850, according to some embodiments. Needle spring assembly 1850 includes a needle tip 1852, a needle cap 1854, spring 1856, and a tube 1858. In assembly 1850, the needle tip 1852 is a hollow needle, that is, it includes a hollow central channel that extends from the tip of the needle 1852 to the opposite side of the needle cap 1854, which connects to one end of the tube 1858. In some embodiments, the hollow needle is used to penetrate the skin, draw the blood, and direct the blood flow into the tube 1858, which in turn may direct the blood flow into channels or chambers such as those described above.

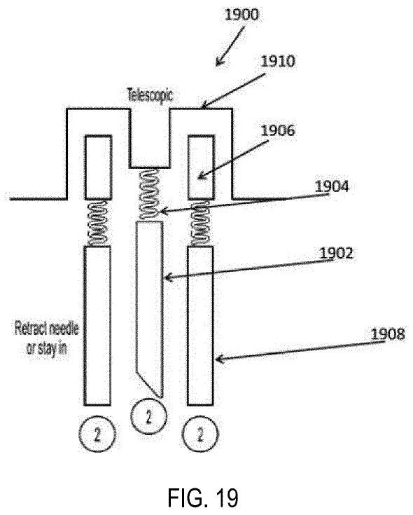

FIG. 19 show a cross section of another needle spring assembly 1900 according to some embodiments. Assembly 1900 includes a blade 1902 that is positioned, within the lumen of a chamber 1903 provided by a substantially spherical wall 1904, which is movable. A cap 1905 and a plurality of springs (collectively referred to as 1906) operably couple the cap to the blade as well as to the distal end of the chamber allow moving the assembly between a retracted position and a deployed position in which the blade can puncture the skin and the proximal end of the chamber is in contact with the skin to surround the puncture site and allow drawing blood from the puncture site through the lumen of the chamber.

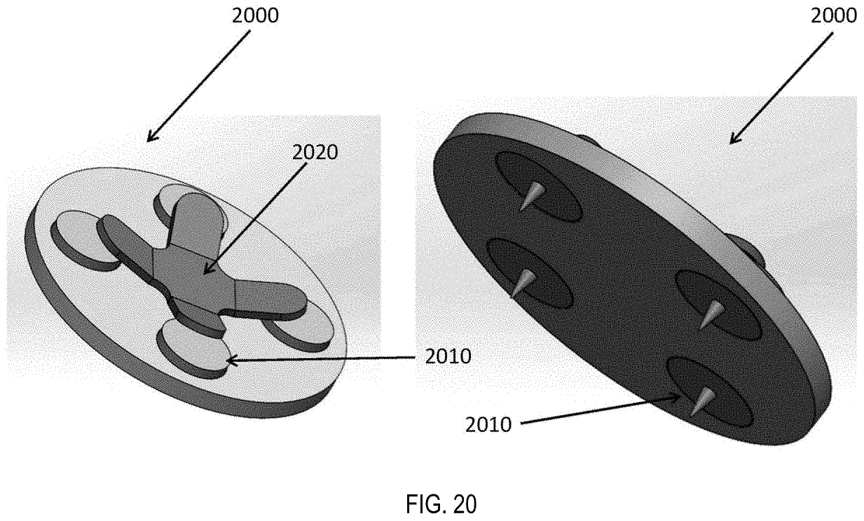

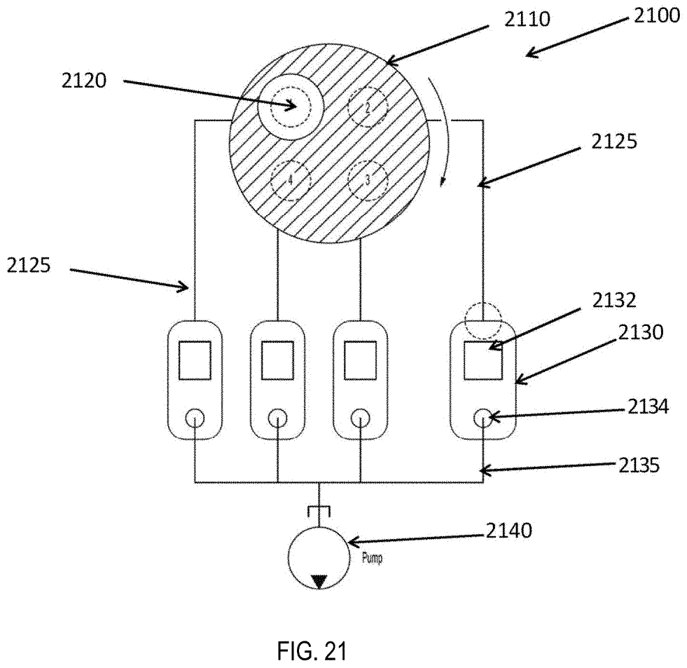

FIG. 20 shows a cantilever type needle assembly 2000 according to some embodiments. Needle assembly 2000 includes four needles 2010 and a cantilever spring assembly 2020. The four needles 2010 are arranged at the four corners of a square located on the needle assembly 2000. The spring assembly 2020 contains 4 cantilever type springs each of them is positioned over one of the four springs 2010. During use, each of the cantilever springs may press down on its associated needle located below it at the desired time in order to, for example, puncture the skin.

Various embodiments may utilize different mechanisms for drawing multiple blood samples and analyzing them at different times. By way of illustration, FIG. 21 shows a high level sketch of a dermal patch 2100, which includes a rotatable dial mechanism for drawing multiple blood samples according to some embodiments. Dermal patch 2100 includes a dial 2110, four needle assemblies 2120, four sensing units 2130, and a pump 2140. Dermal Patch 2100 further includes four fluidic channels 2125 and four vacuum channels 2135.

Dial 2110 includes an actuator 2112 for selectively actuating each of a plurality of needles for puncturing the skin and drawing blood through the puncture site for introduction into one of the four sensing units. In this embodiment, each sensing unit 2130 includes a serum-separation element 2132 and a sensor 2134. Moreover, each fluidic channel 2125 connects one of the needle assemblies 2120 to one of the sensing units 2130. Further, each vacuum channel 2135 connects one of the sensing units 2130 to the pump 2140. A variety of different pumps, such as passive and active pumps, can be employed in different implementations of the dermal patch. Some examples of such pumps include, without limitation, a positive displacement pump, a piezo pump, a capillary pump (i.e., using capillary forces to draw a blood sample). In general, any suitable mechanism for generating a negative pressure for facilitating blood draw from a punctured site may be employed. During operation, dial 2110 may be rotated, for example, clockwise, such that actuator 2112 is positioned over one of needle assemblies 2120. In this position, actuator 2112 may actuate the middle assembly 2112 positioned under the actuator, causing it to puncture the skin such that a blood sample can be collected from the puncture site. The drawn blood may then flow through the corresponding fluidic channel 2125 to the corresponding sensing unit 2130.

In some embodiments, the sensing unit 2130 may include a serum-separation element (e.g., a nitrocellulose strip) that can separate a plasma/serum portion of the blood for analysis by the sensor 2134. Alternatively, the dermal patch may not include a serum-separation element or may include an element that can capture cellular components other than red blood cells, such that the blood sample that reaches the sensor 2130 can be analyzed for detection, for example, of hemoglobin.

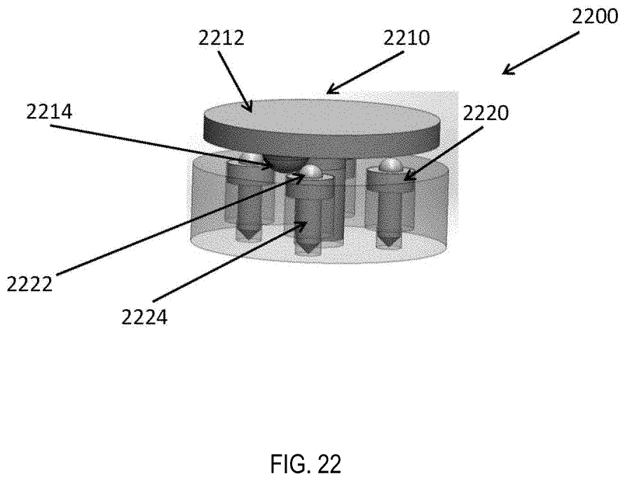

Various embodiments may utilize different mechanisms for actuating the needle assemblies. By way of example, FIG. 22 shows an actuator 2200 utilizing a cam mechanism according to some embodiments for selectively actuating a plurality of needles. Actuator 2200 includes an actuator top section 2210 and four needle assemblies 2220. Top section 2210 includes a dial 2212 and a cam 2214. Each needle assembly 2220 includes a cap 2222 and a needle 2224. During operation, as dial 2212 is rotated, the cam 2214 sequentially engages with the cap 2222 of successive needle assemblies 2220, pressing it down, and thus causing the corresponding needle 2224 to be pushed down, via a spring incorporated in the needle assembly, for puncturing the skin.

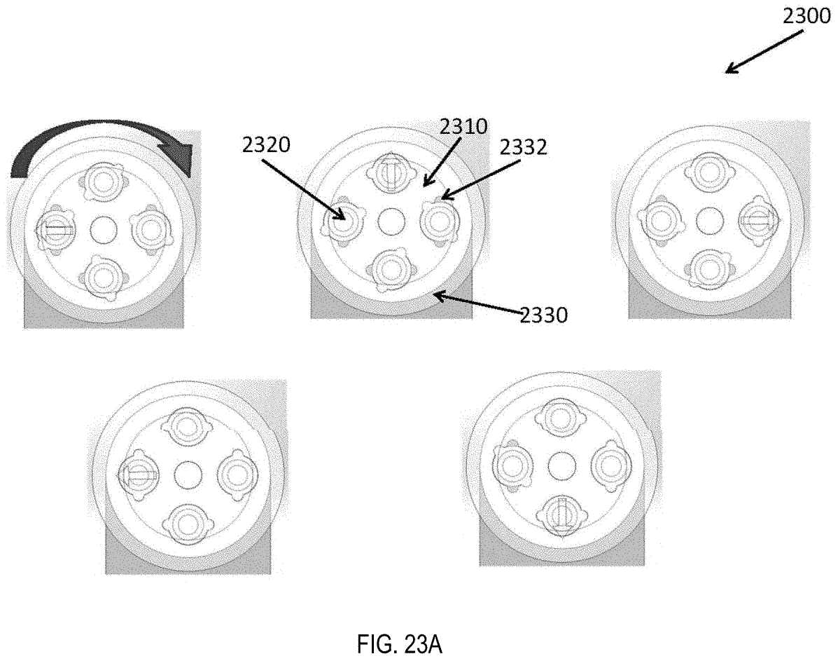

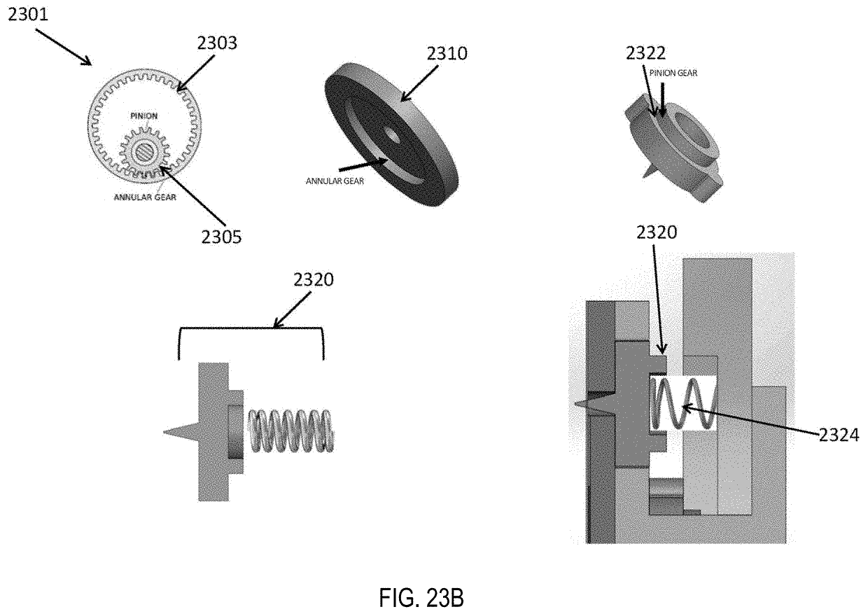

FIGS. 23A and 23B show another actuator 2300 utilizing an annular-pinion mechanism according to some embodiments. Actuator 2300 includes a dial disk 2310, four needle assemblies 2320, and a base 2330. The needle assembly 2320 includes a needle 2322 and a spring 2324. Further, base 2330 includes four holes 2332, each of which are shaped to fit a needle 2322.

The dial disc 2310 and the needle assemblies 2320 together form annular-pinion system. Diagram 2301 demonstrates one such system, in which the outer perimeter of the pinion and the inner perimeter of the annular form two sets of gears that are engaged with each other. When the annular is rotated, for example, clockwise, the pinion also rotates clockwise and further orbits along the inner perimeter of the annular in the clockwise direction.

In the case of the actuator 2300, the outer perimeter of each needle 2322 includes a pinion gear, and the inner perimeter of the dial disc 2310 includes an annular gear. FIG. 23A shows the operation of actuator 2300. As the dial disc 2310 is turned clockwise, the four needle assemblies 2320 one by one align with one of the holes 2332, at which point the spring 2324 of the fitting needle assembly 2320 pushes the corresponding needle 2322 into the hole, thus disengaging the pinion gear of the needle from the annular gear of the dial disc 2310. In this manner, turning the dial disc 2310 causes actuation of the four needle assembly is 2320 at different angles of the rotation.

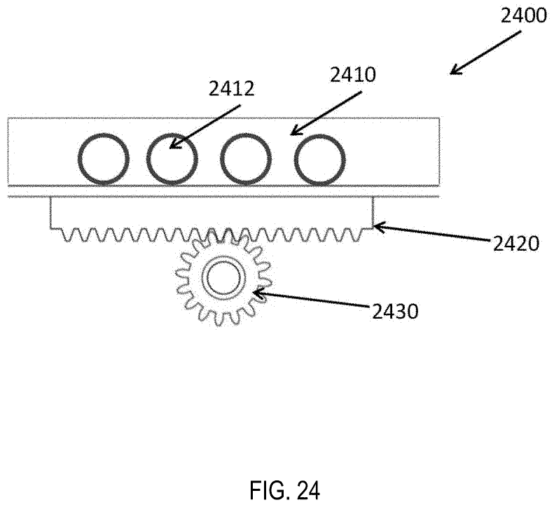

FIG. 24 shows yet another actuator 2400, which utilizes rack and pinion mechanism according to some embodiments. Actuator 2400 includes a needle set 2410, a rack 2420, and a pinion 2430. Needle said 2410 includes four needle assemblies 2412. Unlike the needle assemblies 2320 in actuator 2300, which were located around the circle, needle assemblies 2412 in actuator 2400 are lined up along a line. Further, the needle set 2410 and the rack 2420 are attached to each other.

During operation of actuator 2400, as the pinion 2430 is rotated clockwise, that rotation is transformed into translational motion of the rack 2420 to the right, causing the needle set 2410 to move to the right. This motion can be used to actuate the needle assemblies 2412 at different times.

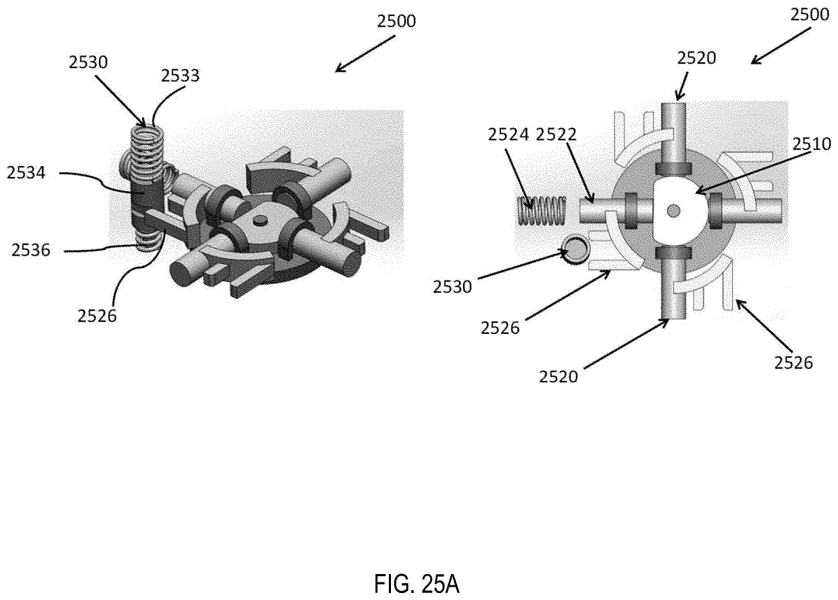

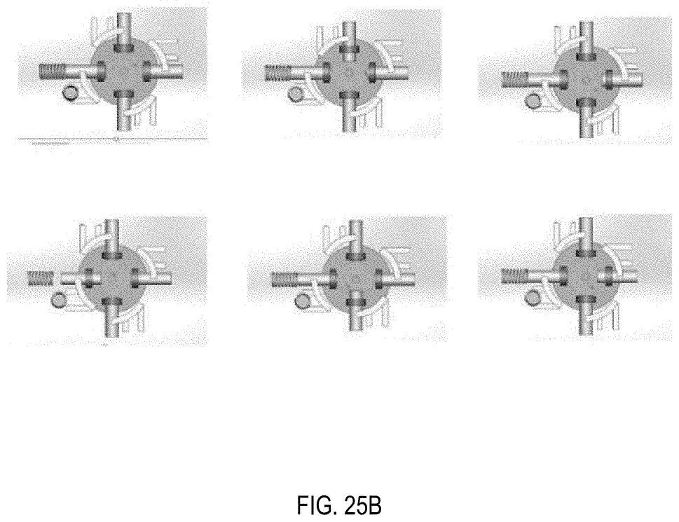

FIGS. 25A and 25B show yet another actuator system 2500, which utilizes another cam mechanism according to some embodiments. Actuator system 2500 includes a cam 2510, four lancet assemblies 2520, and four needle assemblies 2530. Each lancet assembly 2420 includes a lancet shaft 2522, a lancet spring 2524, and two lancet arms 2526. Each needle assembly 2530 includes and upper spring 2532, a needle shaft 2534, and lower spring 2536. In their original state, each needle assembly 2530 is not actuated, that is, the needle shaft is pushed up such that the upper spring 2532 is compressed, and is held in that state by the corresponding needle shaft 2534 which itself is held by the corresponding pair of lancet arms 2526 fit into the grooves on the side of the needle shaft 2534.

FIG. 25B illustrates the operation of the actuator system 2500. As the cam 2510 rotates clockwise, it allows one of the four lancet shaft 2522 to be pushed towards the center by the corresponding lancet spring 2524. As a result, the corresponding pair of lancet arms 2526 are disengaged from the corresponding needle shaft 2534. As a result, the needle shaft is pushed down by the upper spring 2532, causing the needle at the bottom of the needle shaft to move down and puncture the skin. In some embodiments, the combination of the spring constants for the upper spring 2532 and the lower spring 2536 and their locations are selected such that after the needle shaft 2534 punctures the skin, the lower spring 2536 is retracted from the skin.

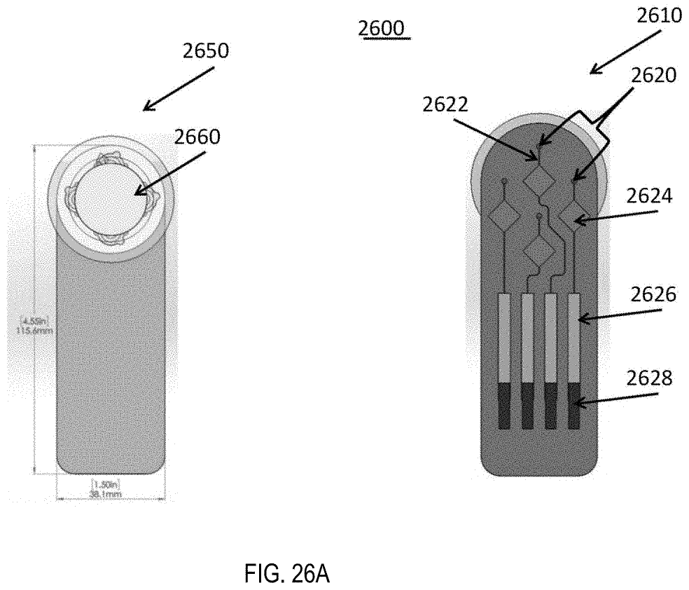

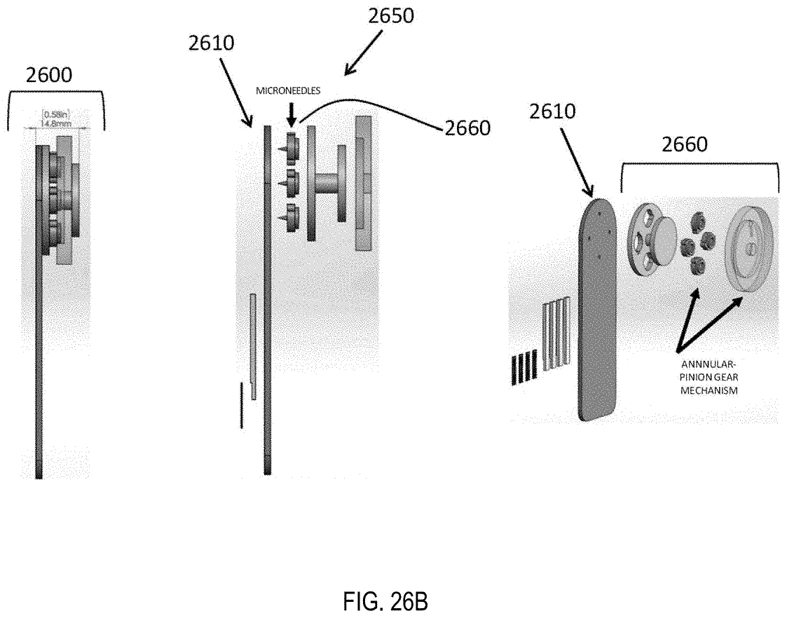

FIGS. 26A and 26B show a dermal patch assembly 2600 according to some embodiments. Dermal Patch assembly 2600 includes a housing having a base 2610 and a cap 2650, which can be releasably engaged with one another to provide an enclosure in which various components of the dermal patch can be disposed. Although in this embodiment the housing of the dermal patch is formed as two separate parts that can be releasably engaged with one another, in other embodiments, the housing of the dermal patch can be formed as a single integral unit.

In this embodiment, the dermal patch includes four blood analysis units 2620, each of which includes a capillary retention valve 2622, a capillary pump 2624 (although in this embodiment, a passive pump is employed, in other embodiments, an active pump may be utilized), a serum-separation element 2626, a sensing unit 2628, and a needle assembly 2629.

The needle assemblies can be actuated, one at a time, via an actuator assembly 2660, which may be, for example, an annular-pinion type actuator similar to actuator 2300 discussed above in relation to FIGS. 23A and 23B to puncture the skin, thereby allowing a blood sample to be drawn from the puncture site and introduced into a respective blood analysis unit.

In this embodiment, the serum-separation element 2626 is a nitrocellulose strip, which can include a wicking element at an end thereof to facilitate the introduction of at least a portion of the blood onto the nitrocellulose strip via wicking action. The nitrocellulose strip can separate a plasma/serum component of the received blood from one or more of its cellular components (e.g., red and white blood cells and platelets) to generate a plasma component or a serum component, which can be received by the sensing unit 2628, which is coupled to the other end of the nitrocellulose strip. Again, in some embodiments, a wicking element coupled to the other end of the nitrocellulose strip can facilitate the transfer of the separated serum component to the sensing unit 2628 associated with a respective blood analysis unit. In this embodiment, the sensing unit 2628 includes a graphene-based sensor, such as those discussed above. It has discovered that although the nitrocellulose strip may not provide as complete separation of the serum component from the cellular elements of the blood as that achievable via traditional methods; nonetheless, the separation achieved via the nitrocellulose strip can significantly enhance the sensitivity and/or specificity of detection of various analytes, including biomarkers, in the resultant serum component, especially in connection with the use of a graphene-based sensor (though other types of sensors, such as electrochemical sensors, may also be employed).

In fact, any suitable sensor, including lateral flow sensors, electrochemical sensors, among others, can be employed in the dermal patch 2600. By way of example, in some embodiment, a lateral flow immunoassay sensor can be employed and the dermal patch may include one or more windows to allow the visual inspection of such a sensor. In some embodiment, prior to the introduction of the serum components (or a whole blood sample in embodiments in which the serum-separation element is not employed) to the sensing unit, isothermal amplification of one or more analytes of interest can be performed, e.g., (LAMP amplification) via an amplification unit that is disposed upstream of the sensing unit. In some such embodiments, such an amplification unit may include lyophilized amplification reagents that can be reconstituted in response to contact with the sample and/or a buffer.

With continued reference to FIGS. 26A and 26B, each of the capillary pumps 2624 receives the blood sample from a puncture site via a fluidic channel 2625 and facilitates the delivery of the blood sample to a respective nitrocellulose strip via another fluidic channel 2627.

A selector dial 2650 allows selectively actuating one of the four needle assemblies, via an actuator 2660, for puncturing the skin and drawing blood from the puncture site. The dermal patch further includes a cartridge 2651 in which a plurality of reservoirs are incorporated, where one or more blood processing reagents, buffers, etc. can be stored for mixing with the received blood sample, e.g., in a manner discussed further below. The dermal patch may include an adhesive layer that allows attaching the patch to a subject's skin.

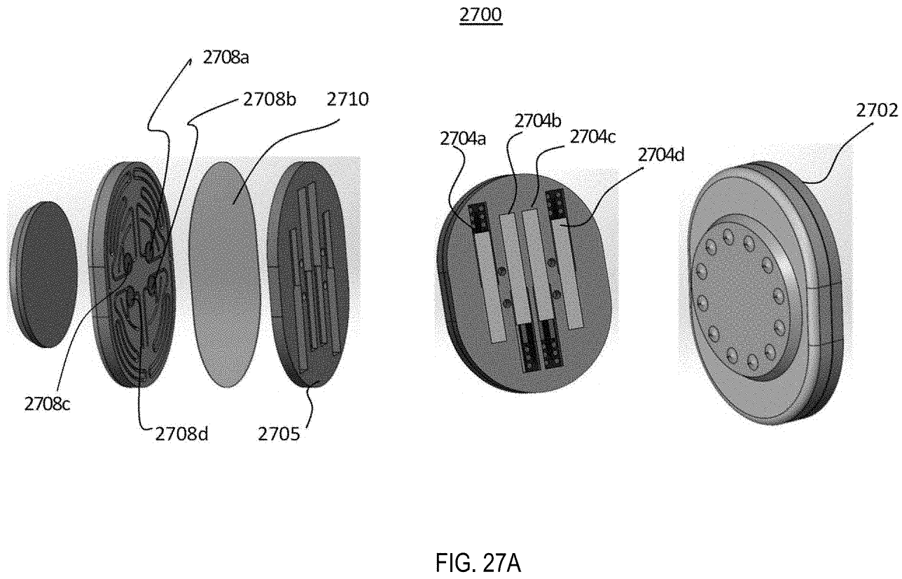

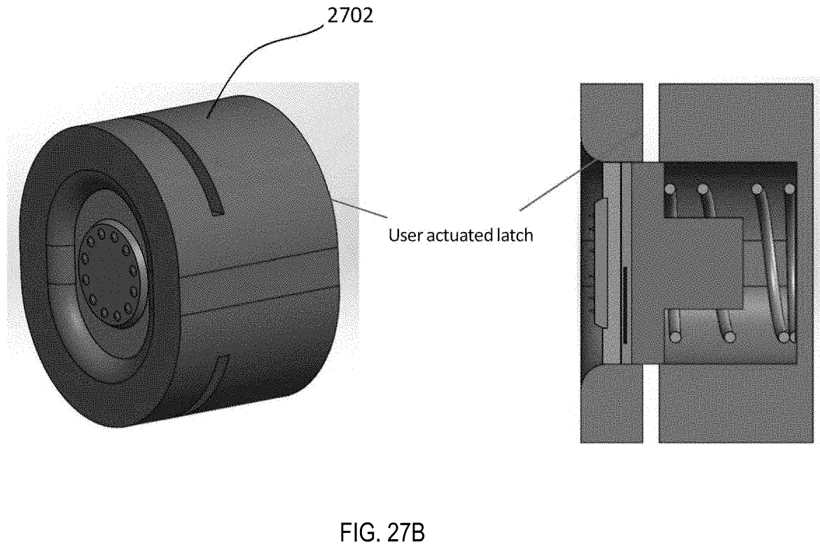

With reference to FIGS. 27A and 27B, a dermal patch 2700 according to another embodiment includes 12 needles in this embodiment, where the needles can be selectively actuated in groups of three, via an actuator 2702. The dermal patch 2700 further includes four sensing units 2704a, 2704b, 2704c, and 2704d, each of which is associated with one group of three needles. The sensing units can be received in a housing 2705.

The dermal patch 2700 further includes a cartridge 2706 having four reservoirs 2708a, 2708b, 2708c, and 2708d (herein collectively referred to as the reservoirs 2708), in each of which one or more blood-processing reagents/buffers can be stored and each of which is associated with one of the sensing units. A seal 2710 separates the cartridge from the sensing units.

FIG. 27B schematically depicts an implementation of the actuator 2702. More specifically, the actuator includes a latch mechanism that can be actuated by a user. Upon moving the latch, the spring may push the needle assembly forward causing it to penetrate the skin.

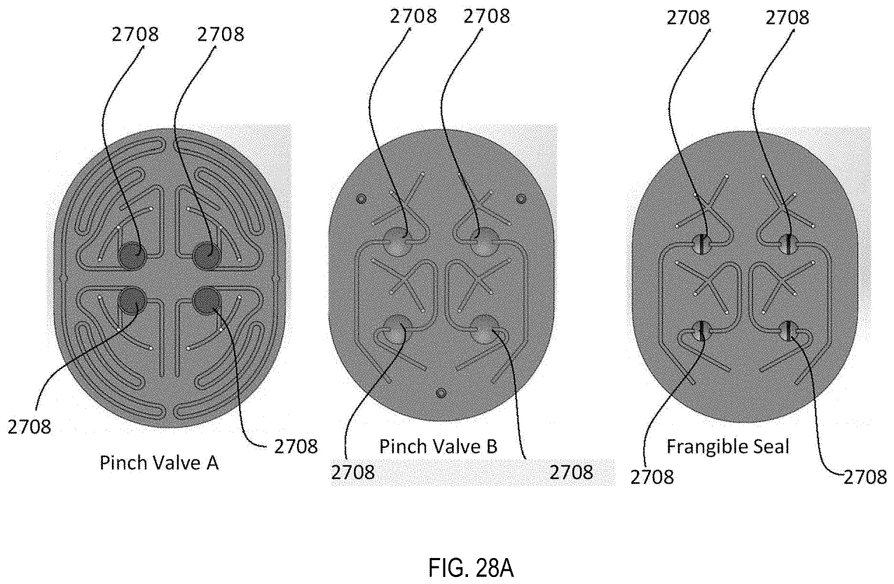

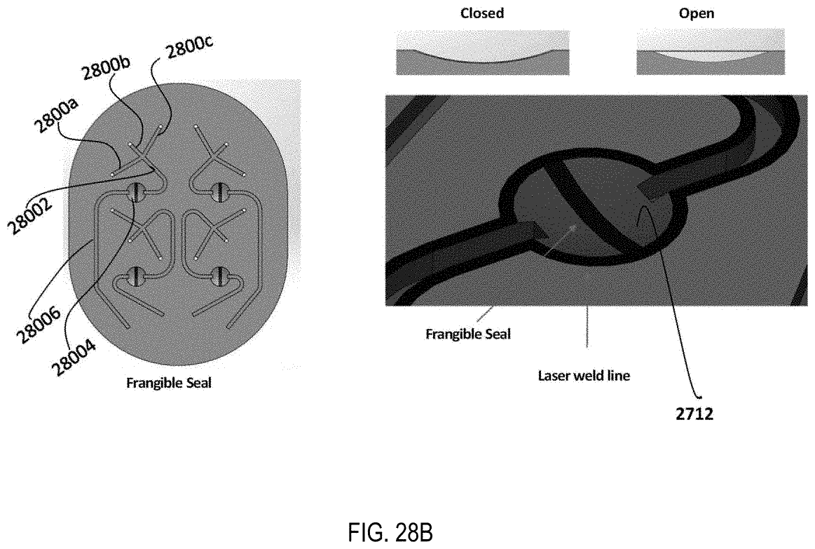

As shown in FIG. 28A, the reagent reservoirs 2708 can be implemented in a variety of different ways. For example, each reservoir can include a pinch valve, which can be activated to release the reagent(s) stored in the respective reservoir for mixing a received blood sample and deliver the mixture via a plurality of fluidic channels to a respective sensing unit.

Alternatively, as shown schematically in FIGS. 28A and 28B, each reservoir can be in the form of a pouch that is sealed via a frangible membrane seal 2712. In some embodiments, the use of the frangible seal membrane advantageously increases the shelf life of the dermal patch by ensuring that the stored reagent(s) remain stable, for example, up to two years or more. In this embodiment, a laser weld seals the frangible seal to the periphery of the pouch.

A pressure can be applied to the frangible seal to burst the seal, thereby releasing the stored reagent(s) into a fluidic channel for mixing with a received blood sample. The mixture of the blood and the processing reagent(s) can be delivered via a fluidic channel to a respective sensing unit. For example, as shown in FIG. 28B, in each blood analysis unit, three fluidic channels 2800a, 2800b, and 2800c receive blood from each of three puncture sites generated by a group of three needles associated with that blood analysis unit. The three fluidic channels merge into a single fluidic channel 2802 in which the blood samples received by the three fluidic channels are combined. The fluidic channel 2802 extends to the reagent reservoir 2804. Upon application of pressure to the frangible membrane of the reagent reservoir 2804 to burst the membrane, the blood mixes with the stored reagent(s) and the mixture is delivered via another fluidic channel 2806 to a sensing unit of the respective blood analysis unit. For example, as discussed above, in some embodiments, the mixture can be delivered to a serum-separating element (e.g., a nitrocellulose strip) of a respective blood analysis unit for separating a serum component of the blood for delivery to a sensor.

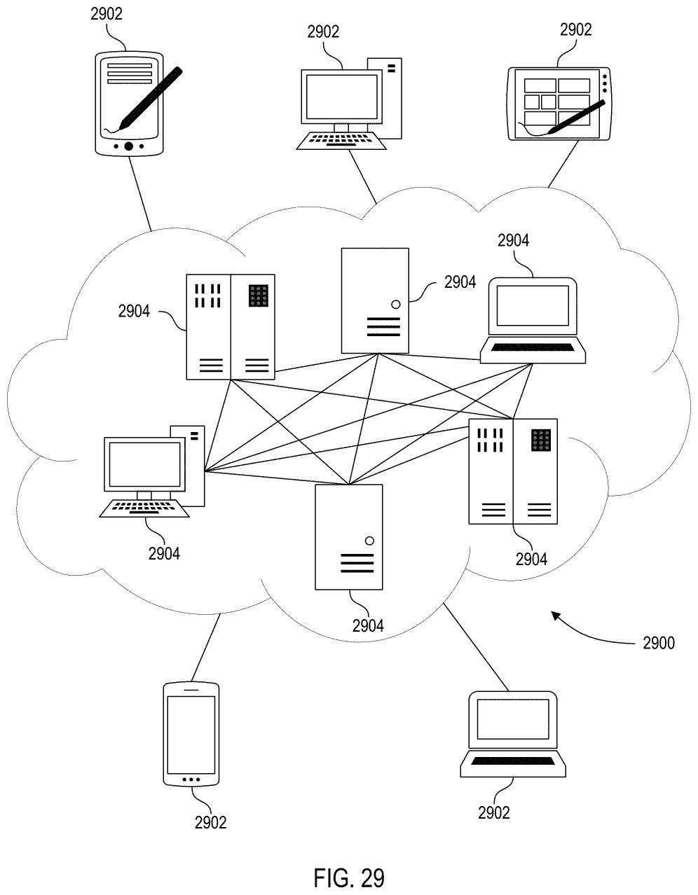

Referring now to FIG. 29, a cloud computing environment 2900 is depicted in accordance with an exemplary embodiment. The cloud computing environment 2900 is connected to one or more user computer systems 2902 and provides network access to shared computer resources (i.e., storage, memory, applications, virtual machines, etc.) to the one or more user computer systems 2902. As depicted in FIG. 29, the cloud computing environment 2900 includes one or more interconnected nodes 2904. Each node 2904 may be a computer system or device with local processing and storage capabilities. The nodes 2904 may be grouped and in communication with one another via one or more networks. This allows the cloud computing environment 2900 to offer software services to the one or more user computer systems 2902 and as such, a user computer system 2902 does not need to maintain resources locally.

In one embodiment, a node 2902 includes the computer system 70001 and as such, includes the computer readable program instructions for carrying out various steps of the methods discussed herein. In these embodiments, a user of a user computer system 2902 that is connected to the cloud computing environment 2900 may cause a node 2904 to execute the computer readable program instructions to carry out various steps of the methods disclosed herein.

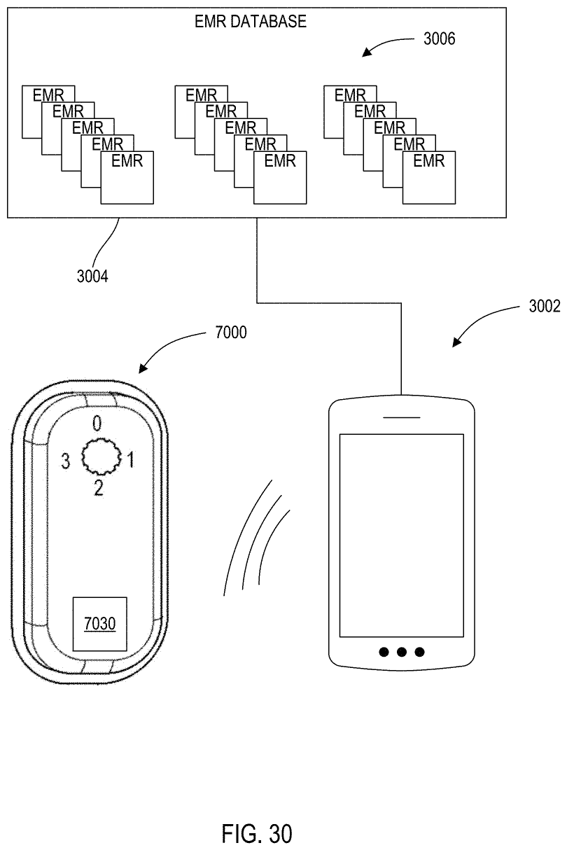

Referring now to FIG. 30 an embodiment of the dermal patch dermal patch 7000 is shown in accordance with an exemplary embodiment.

In this embodiment, a quick response (“QR”) code 7030 is printed onto a top surface of the dermal patch 7000. In this embodiment, a user may install an application stored as computer readable program instructions on a computer system 3002 (i.e., a smartphone, tablet, etc.) and employ a camera of the computer system 3002 to take a photo of the QR code 7030 which is saved in a memory of the computer system 3002. Generally, the computer system 3002 includes same or similar components as the computer system 7000 (i.e., system memory, processor, etc.). In this embodiment, a processor of the computer system 3002 may execute the program instructions associated with the application to retrieve the photograph from the memory.

In some embodiments, the computer system 3002 may be in communication with an electronic medical record (“EMR”) database 3004 via a network connection. The EMR database 3004 includes a plurality of EMRs 3006 each associated with an individual subject. In these embodiments, the instructions associated with the application further cause the processor of the computer system 3002 to analyze the photograph to identify the QR code 7030 and associate the QR code 7030 with an EMR 3006 stored in the EMR database 3004. When a sensor 3000 of the dermal patch 7000 includes a visible readout (e.g., a colorimetric sensor) and the readout is included in the photograph, the processor of the computer system 3002 may further analyze the received photo to evaluate the readout and automatically determine the presence of a target analyte and/or a level of a target analyte based on the readout as previously discussed herein.

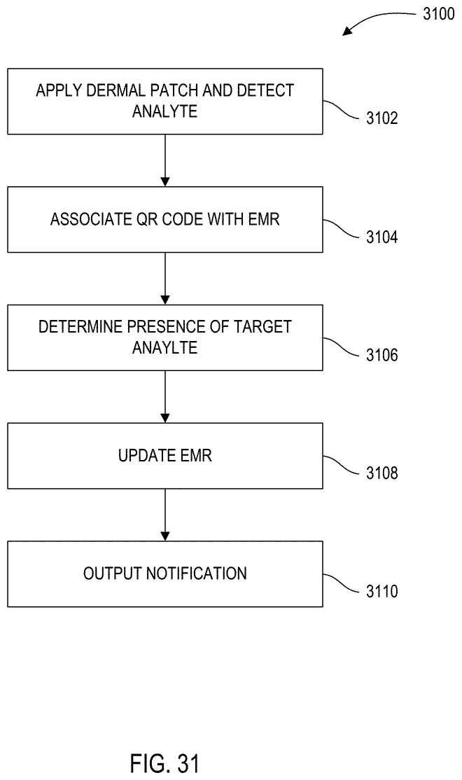

Referring now to FIG. 31, a method 3100 for automatically updating an EMR is shown in accordance with an exemplary embodiment. Steps 3104-3110 of the method 3100 may be stored as computer readable program instructions in a computer readable storage medium (e.g., memory of the computer system 2902 memory of a node 2904, etc.) a processor (e.g., a processor of the computer system 3002, a processor of a node 2904, etc.) executes the computer readable instructions for the steps 3104-3110 of the method 3100.

At 3102, the dermal patch 7000 is applied to the skin of a subject, and is activated to draw a physiological sample form the subject (e.g., a blood sample or a sample of interstitial fluid and the sensor 3000 detects an analyte as previously discussed herein.

At 3104, a user of the computer system 3002 scans the QR code 7030 with a camera of the computer system 3002 and a processor analyzes the QR code 7030 and associates the QR code 7030 with an EMR 3006 as previously discussed herein.

At 3106, the processor analyzes an image of the detector read out (e.g., bands in a lateral flow strip detector) to evaluate the readout of the sensor 3000 and automatically determine whether a target analyte is present in a physiological sample drawn from the subject, and optionally quantify the target analyte if the target analyte is detected in the sample as previously discussed herein.

At 3108, the processor automatically updates the associated EMR to include the determined presence of the target analyte and/or a level of the target analyte. In some embodiments, at 3108, the processor also updates the associated EMR to include the photograph of the QR code and the sensor 3000.

At 3110, the processor outputs a notification indicative of the determined presence of the target analyte and/or the determined level of the target analyte to a display in communication with the processor and/or outputs a notification indicative of the determined presence of the target analyte and/or the determined level of the target analyte to another device (e.g., a physician's smartphone).

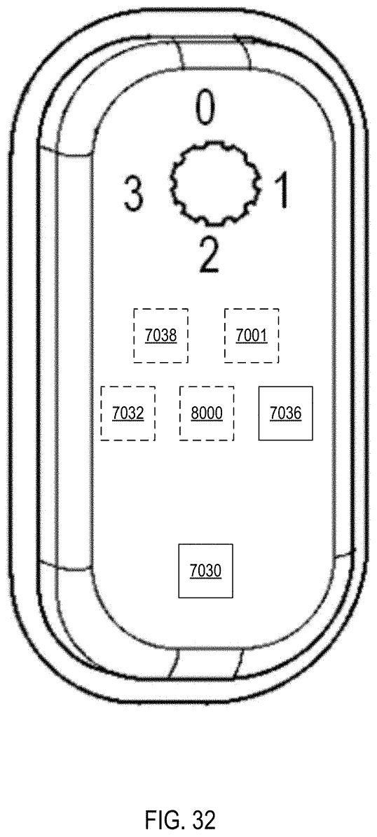

Referring now to FIG. 32, in some embodiments the dermal patch 7000 may further include an electromechanical actuator 7032 that is coupled to and in communication with the selector device 1010 and the actuation mechanism 8000. In this embodiment, the electromechanical actuator is configured to move the selector device 1010 as previously discussed herein and is further configured to cause the actuation mechanism 8000 to move the microneedles 1004 between the retracted and deployed state via rotation of the knob 8002 as previously discussed herein.

The electromechanical actuator 7032 is also connected to and in communication with the computer system 7001. As such the electromechanical actuator 7032 is connected to and in communication with the processor 7008. In some embodiments, the electromechanical actuator 7032 is wirelessly connected to the computer system 7001 and in other embodiments the connection between the electromechanical actuator 7032 and the computer system 7001 is a wired connection. The electromechanical actuator 7032 is configured to move the selector device 1010 and cause the actuation mechanism 8000 to move the microneedles 1004 to the deployed position in response to receiving a signal from the processor 7008.

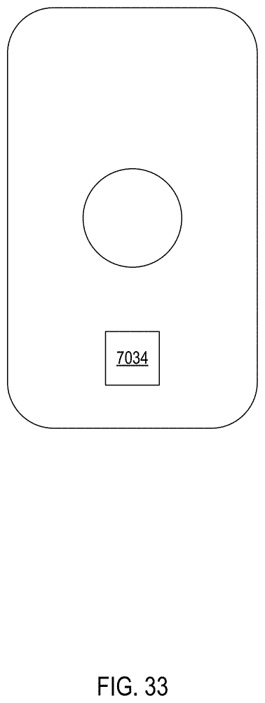

Referring now to FIG. 33, in some embodiments, the dermal patch 7000 may further include a skin sensor 7034 located on a bottom surface of the dermal patch 7000. The skin sensor 7034 is configured to determine when the dermal patch 7000 is adhered to skin of a subject. Stated another way, the skin sensor 7034 is configured to determine when the bottom surface of the dermal patch 7000 contacts skin of the subject. The skin sensor 7034 includes, but is not limited to, optical sensors, infrared sensors, light sensors, temperature sensors, pulse sensors, etc.

The skin sensor 7034 is connected to and in communication with the computer system 7001. As such, the skin sensor 7034 is connected to and in communication with the processor 7008. In some embodiments, the skin sensor 7034 is wirelessly connected to the computer system 7001 and in other embodiments, the connection between the skin sensor 7034 and the computer system 7001 is a wired connection. In response to determining the dermal patch 7000 is adhered to the skin of the subject, the skin sensor 7034 sends a signal to the processor 7008 indicating that the dermal patch 7000 is adhered to the subject.

In some embodiments, in response to receiving the signal indicating that the dermal patch 7000 is adhered to the subject, the processor 7008 sends a signal to the electromechanical actuator 7032 to deploy the needles. In response to receiving the signal to deploy the needles, the electromechanical actuator 7032 causes the actuation mechanism 8000 to move the microneedles 1004 to the deployed position via rotation of the knob 8002. Stated another way, in response to the skin sensor 7034 determining the dermal patch 7000 is adhered to a subject, the processor 7008 automatically causes the dermal patch 7000 to draw a physiological sample as previously discussed herein. In some embodiments, the processor 7008 causes the actuation mechanism 8000 to move the microneedles 1004 to the deployed position after a given amount of time has passed since the skin sensor 7034 determined the dermal patch 7000 was adhered to the subject (e.g., 5 seconds, 10 seconds, 15 seconds, etc.).

As depicted in FIG. 32, the dermal patch 7000 may include an actuation button 7036 and a locking mechanism 7038 (e.g., a pin that prevents mechanical movement of devices coupled to the locking mechanism when in a “locked” position and allows mechanical movement of devices coupled to the locking mechanism when in an “unlocked” position). The actuation button 7036 and the locking mechanism 7038 are connected to and in communication with the actuation mechanism 8000. The locking mechanism 7038 is mechanically coupled to the actuation mechanism 8000 and is movable between a locked state and an unlocked stated. In the locked state, the locking mechanism 7038 prevents the actuation mechanism 8000 from moving the microneedles 1004 to the deployed position, whereas in the unlocked state, actuation mechanism 8000 is capable of moving the microneedles 1004 to the deployed position. The actuation button 7036 is configured to cause the actuation mechanism 8000 to move the microneedles 1004 to the deployed state (e.g., via rotation of the knob 8002) when pressed. As such, after being adhered to a subject, a user may draw a physiological sample from the subject by pushing the actuation button 7036 when the locking mechanism 7038.

In another embodiment, the locking mechanism 7038 is mechanically coupled to the actuation button 7036. In this embodiment, the locking mechanism 7038 does not allow the actuation button 7036 to be depressed when in the locked state. Stated another way, when the locking mechanism 7038 is in the locked state, the actuation button 7036 is not capable of causing the actuation mechanism 8000 to move the microneedles 1004 to the deployed position. When the locking mechanism 7038 is in the unlocked state, the actuation button 7036 may be depressed. Stated another way, a user of the dermal patch 7000 may press the actuation button 7036 to obtain the physiological sample when the locking mechanism 7038 is in the unlocked state.

The electromechanical actuator is coupled to and in communication with the locking mechanism 7038. In one embodiment, after receiving a signal indicating the dermal patch 7000 is adhered to skin of the subject from the skin sensor 7034, the processor 7008 sends a signal to move the locking mechanism 7038 to the unlocked state thereby allowing a user to draw the physiological sample by pushing the actuation button 7036.

As previously discussed herein, a user may employ a camera of the computer system 3002 to scan the QR code 7030. In some embodiments, before scanning the QR code 7030, the previously discussed installed application may require a user to verify their identity (e.g., by entering a password, scanning a fingerprint, etc.). For example, the installed application may require a user to enter a username and password that is associated with an EMR. In response to verifying the identity of the user, the application may unlock thereby allowing the user to scan the QR code 7030. Furthermore, after the application verifies the identity of the user and in response to associating the QR code 7030 with the correct EMR as previously discussed herein, the computer system 3002 may send a signal indicating that the identity of the user has been verified to the processor 7008.

In some embodiments, in response to receiving the signal indicating that the identity of the user has been verified, the processor 7008 sends a signal to the electromechanical actuator 7032 to deploy the microneedles 1004 as previously discussed herein. In the embodiment wherein the dermal patch 7000 includes the locking mechanism 7038, in response to receiving the signal indicating that the identity of the user has been verified, the processor 7008 sends a signal to the electromechanical actuator 7032 to place the locking mechanism 7038 in the unlocked state as previously discussed herein.

In some embodiments, before sending the signal to electromechanical actuator 7032 to deploy the microneedles 1004 or sending the electromechanical actuator 7032 to place the locking mechanism 7038 in the unlocked state, the processor 7008 may only send the signal in response to receiving both the signal indicating that the user identity has been verified and the signal indicating that the dermal patch 7000 has been adhered to skin of the subject as previously discussed herein.

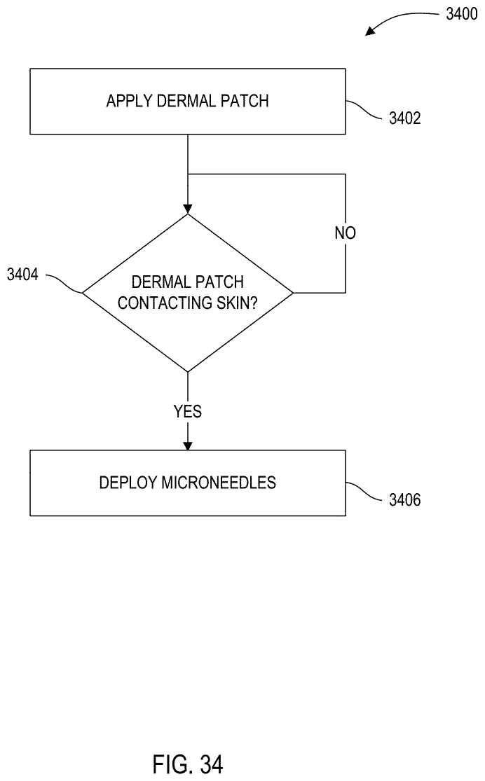

Referring now to FIG. 34, a method 3400 for drawing a physiological sample is shown in accordance with an exemplary embodiment. Steps 3404 and 3406 of the method 3400 may be stored as computer readable program instructions in a computer readable storage medium. A processor executes the computer readable program instructions for the steps 3404 and 3406 of method 3400.

At 3402, the dermal patch 7000 is applied to skin of a subject as previously discussed herein.

At 3404, the skin sensor 7032 determines if the dermal patch 7000 is applied to skin of the subject as previously discussed herein and in response to determining the dermal patch 7000 is adhered to skin of the subject, the skin sensor 7032 sends a signal indicating the dermal patch 7000 is adhered to the processor 7008.

At 3406, in response to receiving the signal indicating the dermal patch 7000 is adhered to the subject, the processor 7008 sends a signal to the electromechanical actuator 7032 to cause the actuation mechanism 8000 to deploy the microneedles 1004 to draw the physiological sample as previously discussed herein.

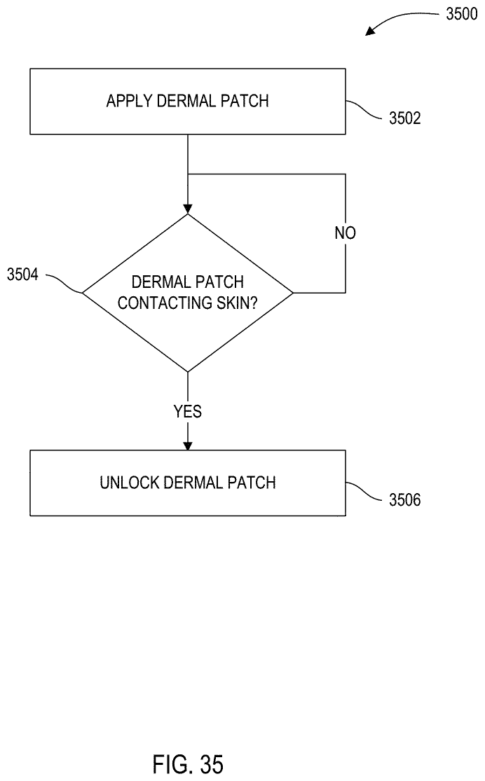

Referring now to FIG. 35, a method 3500 for placing the dermal patch 7000 in a state ready to draw a physiological sample is shown in accordance with an exemplary embodiment. Steps 3504 and 3506 of the method 3500 may be stored as computer readable program instructions in a computer readable storage medium. A processor executes the computer readable program instructions for the steps 3504 and 3506 of method 3500.

At 3502, the dermal patch 7000 is applied to skin of a subject as previously discussed herein.

At 3504, the skin sensor 7032 determines if the dermal patch 7000 is applied to skin of the subject as previously discussed herein and in response to determining the dermal patch 7000 is adhered to skin of the subject, the skin sensor 7032 sends a signal indicating the dermal patch 7000 is adhered to the processor 7008.

At 3506, in response to receiving the signal indicating the dermal patch 7000 is adhered to the subject, the processor 7008 sends a signal to the electromechanical actuator 7032 to place the locking mechanism 7038 in an unlocked position thereby allowing a user to draw a physiological sample as previously discussed herein.

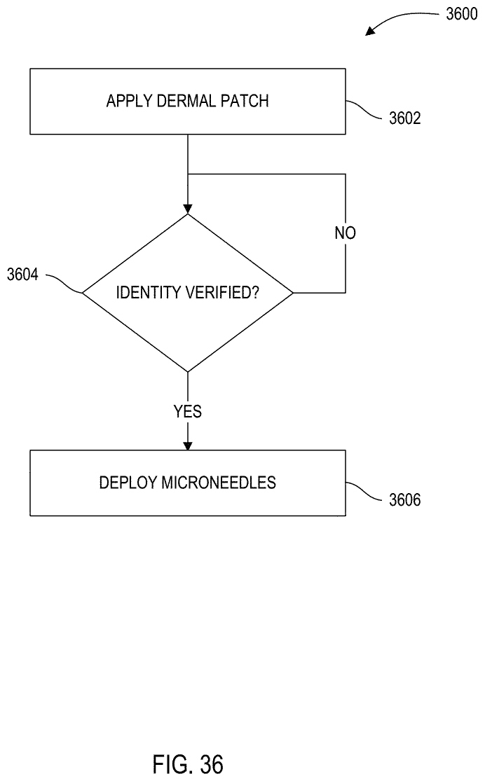

Referring now to FIG. 36, another method 3600 for drawing a physiological sample is shown in accordance with an exemplary embodiment. Steps 3604 and 3606 of the method 3600 may be stored as computer readable program instructions in a computer readable storage medium. A processor executes the computer readable program instructions for the steps 3604 and 3606 of method 3600.

At 3602, the dermal patch 7000 is applied to the skin of a subject as previously discussed herein.

At 3604, a user scans the QR code 7030 and the computer system 3002 verifies the identity of the user as previously discussed herein. In response to verifying the identity of the user, the computer system 3002 sends a signal indicating that the identity of the user has been verified to the processor 7008 as previously discussed herein.

At 3606, in response to receiving the signal indicating that the identity of the user has been verified, the processor 7008 sends a signal to the electromechanical actuator 7032 to cause the actuation mechanism 8000 to deploy the microneedles 1004 to draw the physiological sample as previously discussed herein.

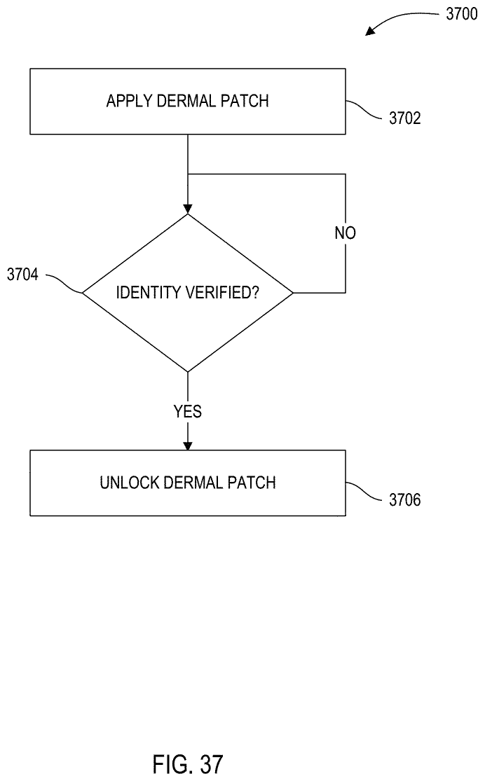

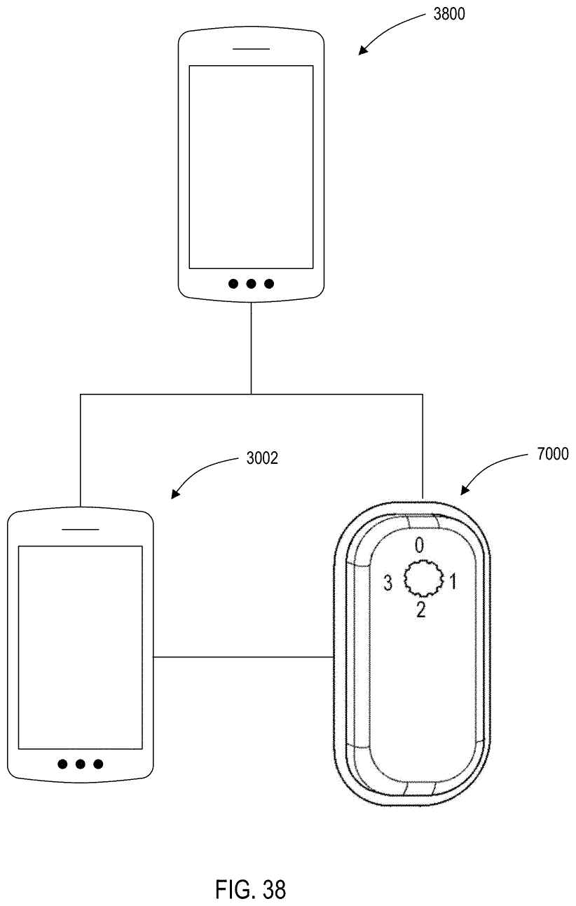

Referring now to FIG. 37, another method 3700 for placing the dermal patch 7000 in a state ready to draw a physiological sample is shown in accordance with an exemplary embodiment. Steps 3704 and 3706 of the method 3700 may be stored as computer readable program instructions in a computer readable storage medium. A processor executes the computer readable program instructions for the steps 3704 and 3706 of method 3700.

At 3702, the dermal patch 7000 is applied to the skin of a subject as previously discussed herein.

At 3704, a user scans the QR code 7030 and the computer system 3002 verifies the identity of the user as previously discussed herein. In response to verifying the identity of the user, the computer system 3002 sends a signal indicating that the identity of the user has been verified to the processor 7008 as previously discussed herein.

At 3706, in response to receiving the signal indicating that the identity of the user has been verified, the processor 7008 sends a signal to the electromechanical actuator 7032 to place the locking mechanism 7038 in an unlocked position thereby allowing a user to draw a physiological sample as previously discussed herein.

Referring now to FIG. 38, a medical professional's computer system 3800 is depicted in accordance with an exemplary embodiment. While FIG. 38 depicts the medical professional's computer system 3800 as a smartphone, in other embodiments the medical professional's computer system 3800 may be another type of computer system (e.g., a tablet, laptop, etc.). As depicted in FIG. 38, the medical professional's computer system 3800 may be connected to and in communication with one of or both of the computer system 3002 and the computer system 7001 (e.g., when the medical professional's computer system 3800, the computer system 3002, and/or the computer system 7001 are connected to a same network).

As previously discussed herein, the processor 7008 may receive a signal indicating that the dermal patch 7000 is adhered to the subject's skin from the skin sensor 7034 or a signal indicating that the identity of the user has been verified. In response to receiving one or both of these signals, the processor 7008 may send a signal indicating that the dermal patch 7000 is ready for operation to a processor of the medical professional's computer system 3800. In some embodiments, after verifying the identity of the user as previously discussed herein, a processor of the computer system 3002 sends a signal indicating that the dermal patch 7000 is ready for operation to the medical professional's computer system 3800.

In response to receiving the signal indicating that the dermal patch 7000 is ready for operation, the processor of the medical professional's computer system 3800 causes a display of the medical professional's computer system 3800 to display a notification indicating the dermal 7000 is ready for operation and displays a GUI with an actuatable icon that when selected by the medical professional sends a signal to deploy the microneedles 1004 or in the embodiment wherein the dermal patch 7000 includes the locking mechanism 7038 sends a signal to unlock the locking mechanism 7038 to the electromechanical actuator 7032 as previously discussed herein.

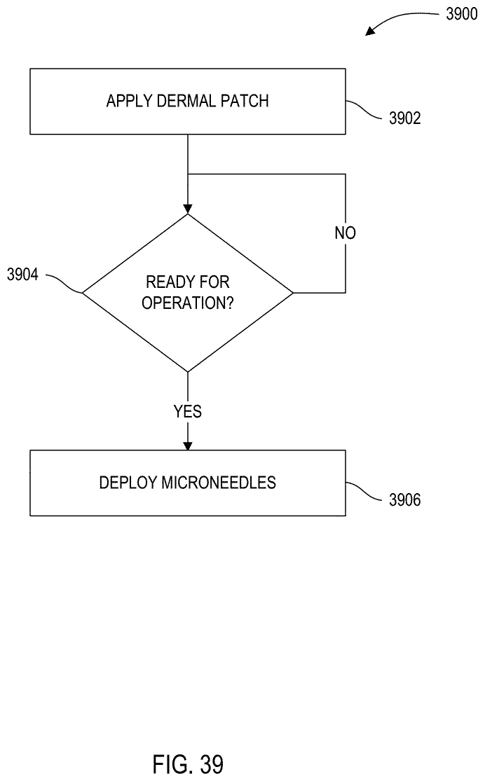

Referring now to FIG. 39, another method 3900 for drawing a physiological sample is shown in accordance with an exemplary embodiment. Steps 3904 and 3906 of the method 3900 may be stored as computer readable program instructions in a computer readable storage medium. A processor executes the computer readable program instructions for the steps 3904 and 3906 of method 3900.

At 3902, the dermal patch 7000 is applied to the skin of a subject as previously discussed herein.

At 3904, the processor 7008 sends a signal indicating the dermal patch 7000 is ready for operation to a medical professional's computer system 3800 in response to verifying an identity of a user and/or in response to determining the dermal patch 7000 is adhered to skin of a subject as previously discussed herein. Furthermore, at 3904, in response to a medical professional selecting an icon displayed in a GUI of a display of the medical professional's computer system 3800, the medical professional's computer system 3800 sends a signal to deploy the microneedles 1004 to the processor 7008 as previously discussed herein.

At 3906, in response to receiving the signal to deploy the microneedles 1004 from the medical professional's computer system 3800 the processor 7008 sends a signal to the electromechanical actuator 7032 to cause the actuation mechanism 8000 to deploy the microneedles 1004 to draw the physiological sample as previously discussed herein.

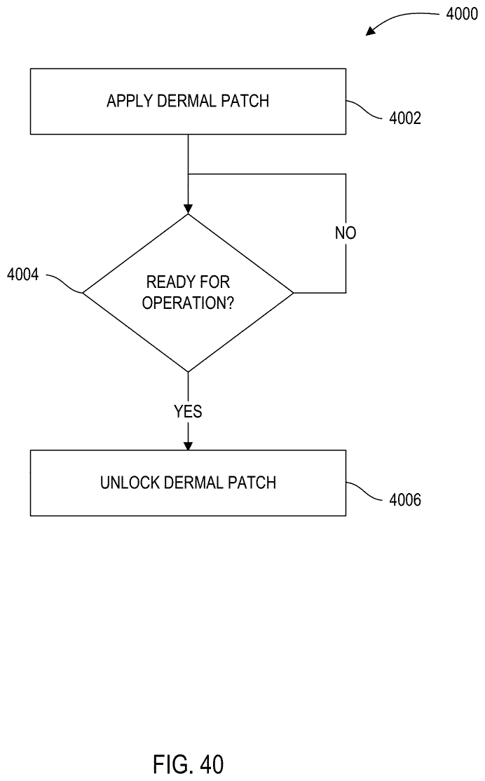

Referring now to FIG. 40, another method 4000 for placing the dermal patch 7000 in a state ready to draw a physiological sample is shown in accordance with an exemplary embodiment. Steps 4004 and 4006 of the method 4000 may be stored as computer readable program instructions in a computer readable storage medium. A processor executes the computer readable program instructions for the steps 4004 and 4006 of method 4000.

At 4002, the dermal patch 7000 is applied to the skin of a subject as previously discussed herein.

At 4004, the processor 7008 sends a signal indicating the dermal patch 7000 is ready for operation to a medical professional's computer system 3800 in response to verifying an identity of a user and/or in response to determining the dermal patch 7000 is adhered to skin of a subject as previously discussed herein. Furthermore, at 4004, in response to a medical professional selecting an icon displayed in a GUI of a display of the medical professional's computer system 3800, the medical professional's computer system 3800 sends a signal to unlock the locking mechanism 7038 as previously discussed herein.

At 4006, in response to receiving the signal to unlock the locking mechanism 7038, the processor 7008 sends a signal to the electromechanical actuator 7032 to place the locking mechanism 7038 in an unlocked position thereby allowing a user to draw a physiological sample as previously discussed herein.

While the methods 3100, 3400, 3500, 3700, 3900, and 4000 include the processor 7008 causing the electromechanically actuator 7032 to deploy the microneedles 1004 or include the processor 7008 causing the electrotechnical actuator 7032 unlocking the dermal patch 7000 in response to receiving one of a signal indicating the dermal patch 7000 is adhered to the subject or a signal indicating that the identity of the user has been verified or in response to receiving a signal from the medical professional's computer system 3800, in other embodiments, the processor 7008 sends the signal deploy the microneedles 1004 or unlock the dermal patch 7000 in response to receiving more than one of the previously recited signals.

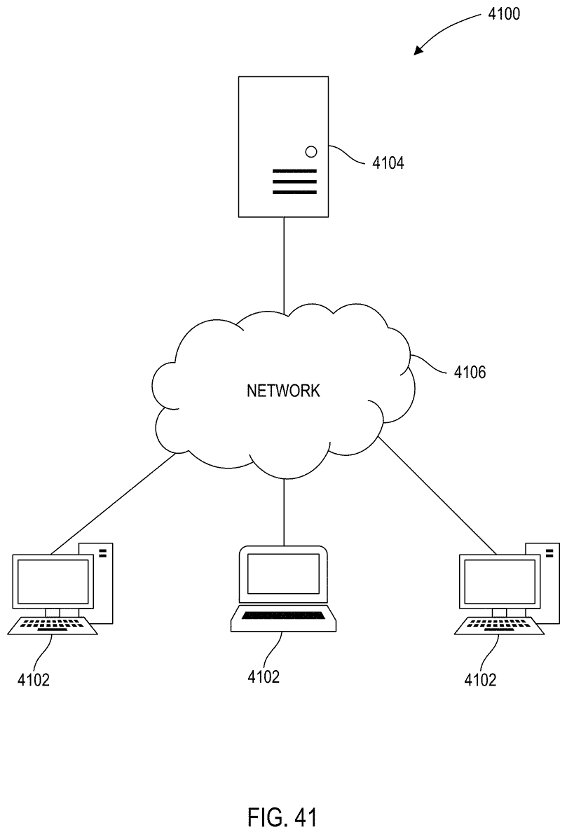

Referring now to FIG. 41, a metaverse network 4100 is shown in accordance with an exemplary embodiment. The metaverse network 4100 includes a plurality of user computer systems 4102, a metaverse server 4104, and a network 4106. In some embodiments, the computer systems 4102 may include one or more of the computer system 7001, the computer system 3002 and the medical professional's computer system 3800. While FIG. 41 depicts the metaverse network 4100 as including three user computer systems 4102 and one metaverse sever 4104, in other embodiments the metaverse network 4100 may include more or less user computer systems 4102 (e.g. 2, 5, 7, etc.) and more than one metaverse server 4104 (e.g., 2, 3, 6, etc.). The user computer systems 4102 are connected to and interface with the metaverse server 4104 via a network (e.g., a local area network (LAN), a wide area network (WAN), a public network (the Internet), etc.).

The metaverse server 4104 hosts a virtual reality environment and/or an augmented reality environment (hereinafter “a metaverse”) with which the users of a computer system 4102 may interact. In one embodiment, a specified area of the metaverse is simulated by a single server instance and the metaverse server 4104 may include a plurality of instances. The metaverse server 3104 may also include a plurality of physics servers configured to simulate and manage interactions, collisions, etc. between characters and objects within the metaverse. The metaverse server 4104 may further include a plurality of storage servers configured to store data relating to characters, media, objects, related computer readable program instructions, etc. for use in the metaverse.

The network 4106 may employ traditional internet protocols to allow communication between user computer systems 4102 and the metaverse server 4104. In some embodiments, the user computer systems 4102 may be directly connected to the metaverse server 4104.

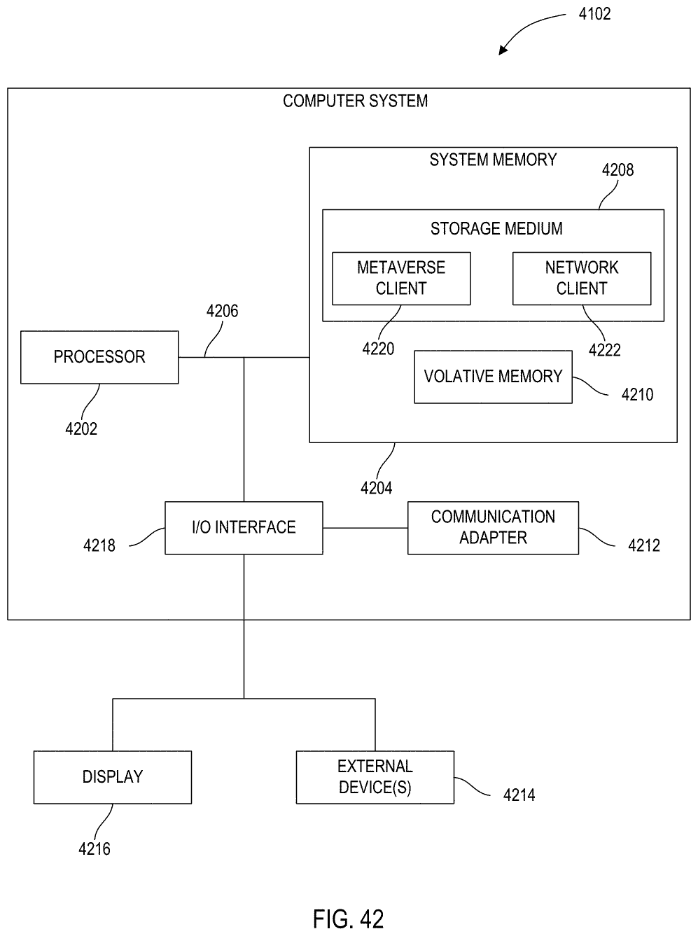

Referring now to FIG. 42 a user computer system 4102 is shown in accordance with an exemplary embodiment. Generally, the user computer system 4102 includes the same or similar components that operate in a same or similar manner as the components of the computer system 7001 (i.e., a processor 4202, system memory 4204, a bus 4206, a computer readable storage medium 4208, volatile memory 4210, a communication adapter 4212, one or more external devices 4214, a display 4216, and an I/O interface 4218). For the sake of brevity, these components are shown, but are not discussed in further detail herein.

The computer system 4102 also includes a metaverse client 4220 and a network client 4222. The metaverse client 4220 and the network client 4222 include computer readable program instructions that may be executed by a processor 4202 of the user computer system 4102. While FIG. 41 depicts the computer readable storage medium 4208 as including the metaverse client 4220 and the network client 4222, in other embodiments the metaverse client 4220 and the network client 4222 may be stored in a different location that is accessible to the processor 4202 (e.g., in a storage element of the cloud computing environment 2900).

When executed, the metaverse client 4220 allows a user of a computer system 4102 to connect to the metaverse server 4104 via the network 4106 thereby allowing a user of the user computer system 4102 to interact with the metaverse provided by the metaverse server 4104. The metaverse client 4220 further allows a user of a user computer system 4102 to interact with other users of other computer systems 4102 that are also connected to the metaverse server 4104.

The network client 4222, when executed by the processor 4202, facilities connection between the user computer system 4102 and the metaverse server 4104 (e.g., by verifying credentials provided by the user). For example, when executed and a user of a computer system 4102 requests to log onto the metaverse server 4104, the network client 4222 maintains a stable connection between the user computer system 4102 and the metaverse server 4104 and handles commands input by a user of a computer system 4102 and handles communications from the metaverse server 4104.

When a user of the user computer system 4102 is logged into the metaverse server 4104, the display 4216 conveys a visual representation of a metaverse provided by the metaverse server 4104. In some embodiments wherein a computer system 4102 is a VR headset and the VR headset includes the display 4216, the metaverse server 4104 provides a three-dimensional (“3D”) environment to the VR headset thereby creating a lifelike environment for the user.

In one embodiment, wherein the computer systems 7001 and 3002 are user computer systems 4102 (and therefore include the metaverse client 4220 and the network client 4222), a user of the dermal patch may log into the metaverse server 4104 by verifying their identity as previously discussed herein. In response to verifying the identity of a user, the computer system 7001 sends a signal indicating the user identity has been verified to the metaverse server 4104 and thereby logging the computer systems 7001 and 3002 into the metaverse.

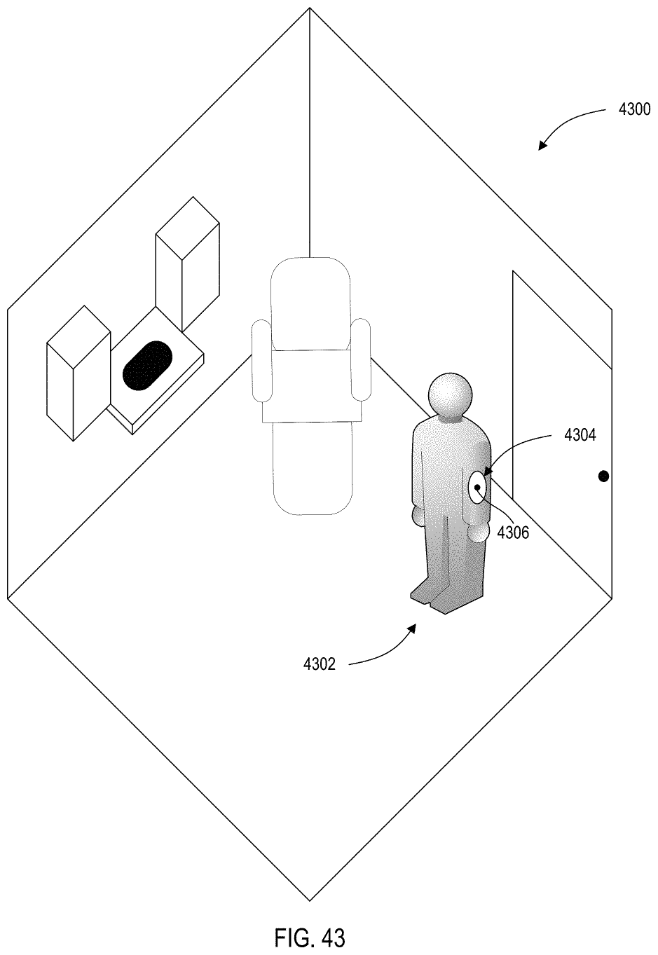

Referring now to FIG. 43, when a user computer system 4102 logs into the metaverse server 4104, the metaverse server 4104 generates a virtual reality environment (or a “metaverse”) 4300 and may generate a subject avatar 4302 corresponding to a user of the dermal patch 7000. In some embodiments, the metaverse 4300 looks like a physician's examination room (e.g., including chairs, examination table, sink, etc.) and may be based on user inputs to create a personalized metaverse 4300. After the subject avatar 4302 is generated, the metaverse server 4104 populates the subject avatar 4302 into the metaverse 4300. In some embodiments, the metaverse server 4104 generates a generic subject avatar 4302 that corresponds to the user and in other embodiments, the subject avatar 4302 has been previously generated by the metaverse based on user inputs. When the subject avatar 4302 is based on user inputs, the avatar may look similar to a subject using the dermal patch 7000.

Furthermore when the computer system 7001 and/or the computer system 3002 is a user computer system 4102 and is logged into the metaverse server 4104, in response to the skin sensor 7034 determining the dermal patch 7000 is contacting skin of the subject and sends a signal to the computer system 7001 or the computer system 3002 indicating the dermal patch 7000 is adhered to the subject as previously discussed herein, the computer system 7001 or the computer system 3002 may send a corresponding signal to the metaverse server 4104. In response to receiving the signal indicating the dermal patch 7000 is adhered to skin of the subject, the metaverse server 4104 generates a virtual dermal patch 4304 on the subject avatar 4302. While the virtual dermal patch 4304 is depicted on an arm of the subject avatar 4302, in other embodiments, the virtual dermal patch 4304 may be depicted as attached to different parts of the subject avatar 4302 (e.g., on a leg of the subject avatar).

The virtual dermal patch 4304 includes an actuatable button 4306. When a user within the metaverse selects the actuatable button 4306, the metaverse server 4104 sends a signal to the processor 7008 of the dermal patch 3006 to deploy the microneedles 1004 or unlock the dermal patch 7000 as previously discussed herein. In response to receiving the signal from the metaverse server 4104, the processor 7008 causes the electromechanical actuator to deploy the microneedles 1004 or unlock the dermal patch 7000 as previously discussed herein. Stated another way, a user in the metaverse 4300 may deploy the microneedles 1004 or unlock the dermal patch 7000 by pushing a button 4306 of a virtual dermal patch 4304. In some embodiments, the actuatable button 4306 may only be actuated by a user of a computer system 4102 with specific login credentials (i.e., a medical professional).

In some embodiments, wherein a user computer system 4102 includes a VR headset that is connected to the metaverse server 4104, a user may view the metaverse 4300 via a display of the VR headset. Furthermore, when the metaverse 4300 includes the avatar 4302 with the virtual dermal patch 4304, the VR headset may track the hands of the user in the VR headset to determine when the user “pushes” (and therefore selects) the actuatable button 4306. In response to determining the user pushed the actuatable button 4306, the VR headset (the user computer system 4102) sends a signal to the metaverse server 4104 indicating a user has selected the actuatable button 4306. In response to receiving this signal, the metaverse server 4104 causes the dermal patch 7000 to deploy the microneedles 1004 or unlock.

In some embodiments, wherein a medical professional logs into the metaverse server 4104 via their login credentials, the metaverse server may populate a corresponding avatar (e.g., a medical professional avatar) into the metaverse 4300. In these embodiments, when the medical professional selects the actuatable button 4306 the metaverse server depicts the medical professional's avatar as interacting with the virtual dermal patch 4304.

Those having ordinary skill in the art will appreciate that various changes may be made to the above embodiments without departing from the scope of the present teachings.

1. A dermal patch, comprising:
at least one needle configured for puncturing a subject's skin so as to allow drawing blood from the subject,
a first blood-transfer fluidic channel for receiving at least a portion of the drawn blood, and
a serum-separation element fluidly coupled to said first blood-transfer fluidic channel for receiving at least a portion of the drawn blood and separating a serum component thereof.

2. The dermal patch of claim 1, wherein said serum-separation element comprises at least one fibrous membrane configured to capture at least a portion of one or more cellular components of the received blood, thereby separating a serum component of the blood.

3. The dermal patch of claim 2, wherein said fibrous membrane comprises nitrocellulose.

4. The dermal patch of claim 3, wherein said fibrous membrane comprises any of a coagulating and an anti-coagulating agent.

5. The dermal patch of claim 2, further comprising a serum-separation fluidic channel in which said fibrous membrane is disposed.

6. The dermal patch of claim 5, wherein said first blood-transfer fluidic channel comprises an inlet for receiving said at least a portion of the blood drawn from the subject and an outlet through which blood exits said first blood-transfer fluidic channel.

7. The dermal patch of claim 6, wherein said serum-separation fluidic channel comprises an inlet in fluid communication with the outlet of said first blood-transfer fluidic channel for receiving at least a portion of the blood exiting said first blood-transfer fluidic channel and an outlet through which the separated serum component exits said serum-separation fluidic channel.

8. The dermal patch of claim 7, further comprising at least one blood-analysis chamber having an inlet in fluid communication with said serum-separation fluidic channel for receiving at least a portion of said serum component.

9. The dermal patch of claim 8, further comprising at least one sensor operably coupled to said blood-analysis chamber for detecting at least one analyte in said serum component.

10. The dermal patch of claim 9, wherein said sensor comprises a graphene-based sensor.

11. The dermal patch of claim 9, wherein said sensor comprises a lateral flow immunoassay sensor.

12. The dermal patch of claim 9, wherein said sensor comprises a chemical sensor.

13. The dermal patch of claim 9, wherein said analyte comprises a biomarker.

14. The dermal patch of claim 9, wherein said serum-separation element comprises at least one wicking element disposed in proximity of the outlet of said serum-separation fluidic channel for capturing at least a portion of the separated serum component and delivering at least a portion of said captured serum component to a sensing element of said sensor.

15. The dermal patch of claim 1, wherein said serum component comprises any of a plurality of red blood cells and a plurality of white blood cells.

16. The dermal patch of claim 15, wherein said concentration of the red blood cells in said separated serum component is half of that in the whole blood.

17. The dermal patch of claim 1, further comprising a housing for containing a plurality of microneedles, said first blood-transfer fluidic channel and said serum-separation element.

18. The dermal patch of claim 17, wherein said microneedles are movable between a retracted position and a deployed position.

19. The dermal patch of claim 18, wherein said housing comprises a chamber for receiving said microneedles in the retracted position.

20. The dermal patch of claim 19, further comprising a mechanism for moving said microneedles between said retracted and said deployed positions.

21. The dermal patch of claim 1, further comprising at least one whole-blood receiving well for receiving a portion of the blood drawn from the subject.

22. The dermal patch of claim 21, further comprising a mechanism operably coupled to said at least one well for spatially distributing the blood received in said well into a mono-layer blood smear.

23. The dermal patch of claim 22, wherein said blood receiving well comprises at least one transparent window to allow acquisition of an image of said monolayer blood smear for counting at least one cellular component of the blood.

24. The dermal patch of claim 22, further comprising a blood distribution fluidic device having an inlet in fluid communication with said first blood transfer fluidic channel for receiving at least a portion of said drawn blood, said blood distribution fluidic device having at least two outlets one of which is in fluid communication with said at least one well and another one of which is in fluidic communication with said serum-separation element for distributing said received blood between said at least one well and said serum-separation element.

25. A dermal patch, comprising:
a plurality of sensing units,
a plurality of needles, wherein each of said needles is associated with one of said sensing units and is configured for puncturing the skin for drawing blood for introduction into the respective sensing unit, and
at least one serum-separation element associated with at least one of said sensing units for receiving blood and separating a serum component of the blood for introduction into said at least one of the sensing units.

26. The dermal patch of claim 25, wherein said at least one serum-separation element comprises a plurality of serum-separation elements each of which is associated with one of said sensing units.

27. The dermal patch of claim 25, further comprising at least one pump for facilitating transfer of the blood to said at least one sensing unit.

28. The dermal patch of claim 27, wherein said at least one pump comprises a plurality of pumps each of which is configured to facilitate transfer of blood drawn via one of said needles to a sensing unit associated with that needle.

29. The dermal patch of claim 27, wherein said at least one pump comprises a passive pump.

30. The dermal patch of claim 27, wherein said at least one pump comprises an active pump.
